(19) United States
(12) Patent Application Publication
(10) Pub. No.: US 2014/0221676 A1
(43) Pub. Date: Aug. 7, 2014

(54) COMPOSITIONS AND METHODS FOR TREATING AND/OR PREVENTING CARDIOVASCULAR DISEASE

(71) Applicant: Amarin Pharmaceuticals Ireland Limited, Dublin (IE)

(72) Inventors: Rene Braeckman, Richboro, PA (US); William Stirtan, East Lyme, CT (US); Paresh Soni, Mystic, CT (US)

(73) Assignee: Amarin Pharmaceuticals Ireland Limited, Dublin (IE)

(21) Appl. No.: 14/173,076

(22) Filed: Feb. 5, 2014

Related U.S. Application Data
(60) Provisional application No. 61/761,398, filed on Feb. 6, 2013.
(60) Provisional application No. 61/830,463, filed on Jun. 3, 2013.

(51) Int. Cl. C07C 69/587

(52) U.S. Cl. CPC C07C 69/587
USPC 554/224

(57) ABSTRACT

In various embodiments, the present invention provides pharmaceutical compositions comprising fatty acids and methods for treating subjects using same.

### PRIORITY CLAIM

This application claims priority to U.S. provisional application No. 61/761,398, filed on Feb. 6, 2013, and U.S. provisional application No. 61/830,463, filed on Jun. 3, 2013, the entire contents of each of which are incorporated herein by reference and relied upon.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dyslipidemia, congestive heart failure and stroke. A need exists for improved treatments for cardiovascular-related diseases and disorders.

### SUMMARY

In various embodiments, the present invention provides pharmaceutical compositions and methods of using such compositions to increase plasma, serum and/or red blood cell (RBC) EPA levels and/or to treat or prevent cardiovascular-related diseases.

In one embodiment, a pharmaceutical composition according to the present disclosure comprises about 1 g of ethyl eicosapentaenoate and provides a mean plasma Cₘₐₓ of about 154.9+/−49.4 μg/mL, a mean plasma AUC₀₋₂₄ₕ of about 2907+/−1160 μg·h/mL, a median plasma Tₘₐₓ of about 5 hours, and a mean plasma T_{1/2} of about 75.1+/−46.5 hours of ethyl eicosapentaenoate when administered orally twice per day to a human subject.

In another embodiment, a pharmaceutical composition comprises about 2 g of ethyl eicosapentaenoate and provides a mean plasma Cₘₐₓ of about 347.2+/−112.5 μg/mL, a mean plasma AUC₀₋₂₄ₕ of about 6519+/−1963 μg·h/mL, a median plasma Tₘₐₓ of about 5 hours, and a mean plasma T_{1/2} of about 89.3+/−42.0 hours of ethyl eicosapentaenoate when administered orally twice per day to a human subject.

These and other embodiments of the present invention will be disclosed in further detail herein below.

### BRIEF DESCRIPTION OF THE DRAWINGS

FIG. 1 depicts the distribution and disposition of subjects among four dosing groups, referred to herein as Groups 1 to 4.

FIG. 2 depicts the mean total plasma eicosapentaenoic acid concentration over time for dosing Groups 1 to 4. Bars indicate one standard deviation at each point.

FIG. 3 depicts the mean total plasma eicosapentaenoic acid trough concentration (Cₘᵢₙ) over time for dosing Groups 1 to 4. Bars indicate one standard deviation at each point.

FIG. 4A depicts the mean total eicosapentaenoic acid plasma AUC₀₋₂₄ₕ measured 28 days after onset of dosing for dosing Groups 1 to 4. FIG. 4B depicts the mean unesterified eicosapentaenoic acid plasma AUC₀₋₂₄ₕ measured 28 days after onset of dosing for dosing Groups 1 to 4. FIG. 4C depicts the mean total eicosapentaenoic acid red blood cell (RBC) AUC₀₋₂₄ₕ measured 28 days after onset of dosing for dosing Groups 1 to 4. Bars in FIGS. 4A to 4C indicate one standard deviation.

FIG. 5A depicts the mean total eicosapentaenoic acid plasma Cₘₐₓ measured 28 days after onset of dosing for dosing Groups 1 to 4. FIG. 5B depicts the mean unesterified eicosapentaenoic acid plasma Cₘₐₓ measured 28 days after onset of dosing for dosing Groups 1 to 4. FIG. 5C depicts the mean total eicosapentaenoic acid red blood cell (RBC) Cₘₐₓ measured 28 days after onset of dosing for dosing Groups 1 to 4. Bars in FIGS. 5A to 5C indicate one standard deviation.

FIG. 6 depicts median triglyceride percent change from baseline (y-axis) vs. mean plasma EPA concentration percent change from baseline (x-axis) for subjects administered placebo, EPA 2 g per day and EPA 4 grams per day. Bars in FIG. 6 indicate 95% confidence intervals for both axes.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

In one embodiment, the invention provides pharmaceutical compositions comprising eicosapentaenoic acid or a derivative thereof. In one embodiment, such compositions comprise eicosapentaenoic acid, or a pharmaceutically acceptable ester, derivative, conjugate or salt thereof, or mixtures of any of the foregoing, collectively referred to herein as “EPA.” The term “pharmaceutically acceptable” in the present context means that the substance in question does not produce unacceptable toxicity to the subject or interaction with other components of the composition.

In one embodiment, the EPA comprises all-cis eicosa-5,8,11,14,17-pentaenoic acid. In another embodiment, the EPA comprises an eicosapentaenoic acid ester. In another embodiment, the EPA comprises a C₁-C₅ alkyl ester of eicosapentaenoic acid. In another embodiment, the EPA comprises eicosapentaenoic acid ethyl ester, eicosapentaenoic acid methyl ester, eicosapentaenoic acid propyl ester, or eicosapentaenoic acid butyl ester. In another embodiment, the EPA comprises all-cis eicosa-5,8,11,14,17-pentaenoic acid ethyl ester.

In another embodiment, the EPA is in the form of ethyl-EPA, lithium EPA, mono-, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree.

In another embodiment, the composition is present in a composition or dosage unit (e.g., a capsule) in an amount of about 50 mg to about 5000 mg, about 75 mg to about 2500 mg, or about 100 mg to about 1000 mg.

In another embodiment, EPA comprises at least 70%, at least 80%, at least 90%, at least 95%, at least 96%, at least 97%, at least 98%, at least 99%, or 100%, by weight, of all fatty acids present in a composition according to the invention.

In another embodiment, a composition useful in accordance with the invention contains less than 10%, less than 9%, less than 8%, less than 7%, less than 6%, less than 5%, less than 4%, less than 3%, less than 2%, less than 1%, less than 0.5% or less than 0.25%, by weight of the total composition or by weight of the total fatty acid content, of any fatty acid or derivative thereof other than EPA. Illustrative examples of a “fatty acid other than EPA” include linolenic acid (LA), arachidonic acid (AA), docosahexaenoic acid (DHA), alpha-linolenic acid (ALA), stearadonic acid (STA), eicosatrienoic acid (ETA) and/or docosapentaenoic acid (DPA). In another embodiment, a composition useful in accordance with the invention contains about 0.1% to about 4%, about 0.5% to about 3%, or about 1% to about 2%, by weight, of total fatty acids other than EPA and/or DHA.

In another embodiment, a composition in accordance with the invention has one or more of the following features: (a) eicosapentaenoic acid ethyl ester represents at least about 96%, at least about 97%, or at least about 98%, by weight, of all fatty acids present in the composition; (b) the composition contains not more than about 4%, not more than about 3%, or not more than about 2%, by weight, of total fatty acids other than eicosapentaenoic acid ethyl ester; (c) the composition contains not more than about 0.6%, not more than about 0.5%, or not more than about 0.4% of any individual fatty acid other than eicosapentaenoic acid ethyl ester; (d) the composition has a refractive index (20° C.) of about 1 to about 2, about 1.2 to about 1.8 or about 1.4 to about 1.5; (e) the composition has a specific gravity (20° C.) of about 0.8 to about 1.0, about 0.85 to about 0.95 or about 0.9 to about 0.92; (e) the composition contains not more than about 20 ppm, not more than about 15 ppm or not more than about 10 ppm heavy metals, (f) the composition contains not more than about 5 ppm, not more than about 4 ppm, not more than about 3 ppm, or not more than about 2 ppm arsenic, and/or (g) the composition has a peroxide value of not more than about 5 meq/kg, not more than about 4 meq/kg, not more than about 3 meq/kg, or not more than about 2 meq/kg.

In another embodiment, the invention provides a composition comprising, consisting essentially of, or consisting of at least 95%, 96% or 97%, by weight, ethyl eicosapentaenoate, about 0.2% to about 0.5% by weight ethyl octadecatetraenoate, about 0.05% to about 0.25% by weight ethyl nonadecapentaenoate, about 0.2% to about 0.45% by weight ethyl arachidonate, about 0.3% to about 0.5% by weight ethyl eicosatetraenoate, and about 0.05% to about 0.32% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g., tocopherol) or other impurities in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell.

In another embodiment, the invention provides a composition comprising, consisting of or consisting essentially of at least 96% by weight ethyl eicosapentaenoate, about 0.22% to about 0.4% by weight ethyl octadecatetraenoate, about 0.075% to about 0.20% by weight ethyl nonadecapentaenoate, about 0.25% to about 0.40% by weight ethyl arachidonate, about 0.3% to about 0.4% by weight ethyl eicosatetraenoate and about 0.075% to about 0.25% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g., tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell.

In another embodiment, the invention provides a composition comprising, consisting of, or consisting essentially of at least 96%, 97% or 98%, by weight, ethyl eicosapentaenoate, about 0.25% to about 0.38% by weight ethyl octadecatetraenoate, about 0.10% to about 0.15% by weight ethyl nonadecapentaenoate, about 0.25% to about 0.35% by weight ethyl arachidonate, about 0.31% to about 0.38% by weight ethyl eicosatetraenoate, and about 0.08% to about 0.20% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g., tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell.

In another embodiment, the invention provides a method of increasing serum, plasma and/or red blood cell (RBC) EPA levels comprising administering a composition as described herein to a subject in need of such treatment. In one embodiment, upon orally administering a composition as set forth herein to a subject for a period of at least about 5, about 10, about 15, about 20, about 25, about 30, about 35, about 40, about 42, about 45 or about 50 days, the subject exhibits at least about a 2-fold, at least about a 3-fold, at least about a 3.5-fold, at least about a 3.75-fold or at least about a 4-fold increase or change (final absolute EPA level divided by baseline EPA level) in serum, plasma and/or RBC EPA. In one embodiment, the method comprises a step of identifying a patient in need of an increase in serum, plasma and/or red blood cell (RBC) EPA prior to said administration step. In a related embodiment, the subject has a baseline EPA plasma, serum and/or RBC level not greater than about 50 μg/g. In another embodiment, the subject is provided with about 2 g to about 4 g per day of a composition as described herein. In another embodiment, upon administering the composition to the subject as per above, the subject exhibits a decrease in DHA, AA and/or DGLA plasma, serum and/or RBC levels. In another embodiment, upon administering the composition to the subject as per above, the subject exhibits an increase in DPA plasma, serum and/or RBC levels. In still another embodiment, upon administering the composition to the subject as per above, DHA plasma, serum and/or RBC levels decrease by at least 15%, DGLA plasma, serum and/or RBC levels decrease by at least 30%, AA plasma, serum and/or RBC levels decrease by at least 20%, and/or DPA plasma, serum and/or RBC levels increase by greater than 130%.

In another embodiment, the invention provides a method of increasing serum, plasma and/or red blood cell (RBC) EPA levels comprising administering a composition as described herein to a subject in need of increased serum, plasma and/or RBC EPA levels. In a related embodiment, upon administering the composition to the subject for a period of at least about 5, about 10, about 15, about 20, about 25, about 30, about 35, about 40, about 42, about 45, or about 50 days, the subject exhibits at least about a 100%, at least about a 150%, at least about a 200%, at least about a 250%, at least about a 300%, at least about a 350% or at least about a 400% increase (change in EPA level from baseline divided by baseline EPA level) in plasma, serum and/or RBC EPA levels compared to baseline. In a related embodiment, the subject has a baseline EPA plasma, serum and/or RBC level not greater than about 50 μg/g. In another embodiment, the subject is provided with about 2 g to about 4 g per day of a composition as described herein. In another embodiment, upon administering the composition to the subject as per above, the subject exhibits a decrease in DHA, AA and/or DGLA plasma, serum and/or RBC levels. In another embodiment, upon administering the composition to the subject as per above, the subject exhibits an increase in DPA plasma, serum and/or RBC levels. In still another embodiment, upon administering the composition to the subject as per above, DHA plasma, serum and/or RBC levels decrease by at least 15%, DGLA plasma, serum and/or RBC levels decrease by at least 30%, AA plasma, serum and/or RBC levels decrease by at least 20%, and/or DPA plasma, serum and/or RBC levels increase by greater than 130%.

In a related embodiment, upon orally administering about 2 to about 4 g per day of a composition as described herein to a subject for a period of at least about 5, about 10, about 15, about 20, about 25, about 30, about 35, about 40, about 45 or about 50 days, the subject exhibits at least about a 10 μg/g increase, at least about a 15 μg/g increase, at least about a 20 μg/g increase, at least about a 25 μg/g increase, at least about a 30 μg/g increase, at least about a 35 μg/g increase, at least about a 40 μg/g increase, at least about a 45 μg/g increase, at least about a 50 μg/g increase, at least about a 75 μg/g increase, at least about a 100 μg/g increase, or at least about a 150 μg/g increase in serum, plasma and/or RBC EPA compared to baseline. In another embodiment, upon administering the composition to the subject as per above, the subject exhibits a decrease in DHA, AA and/or DGLA plasma, serum and/or RBC levels. In another embodiment, upon administering the composition to the subject as per above, the subject exhibits an increase in DPA plasma, serum and/or RBC levels. In still another embodiment, upon administering the composition to the subject as per above, DHA plasma, serum and/or RBC levels decrease by at least 15%, DGLA plasma, serum and/or RBC levels decrease by at least 30%, AA plasma, serum and/or RBC levels decrease by at least 20%, and/or DPA plasma, serum and/or RBC levels increase by greater than 130%.

In another embodiment, the subject has not been on an omega-3 fatty acid therapy or supplement for at least 2 weeks, 3 weeks, 4 weeks, 6 weeks or 12 weeks prior to initiating therapy as described herein.

In one embodiment, the invention provides a method for treatment and/or prevention of cardiovascular-related diseases comprising administering to a subject in need of such treatment or prevention a composition as set forth herein. The term “cardiovascular-related disease” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the present invention provides a method of blood lipid therapy comprising administering to a subject or subject group in need thereof a pharmaceutical composition as described herein. In another embodiment, the subject or subject group has hypertriglyceridemia, hypercholesterolemia, mixed dyslipidemia, borderline high/high triglycerides (e.g., about 200 to about 499 mg/dl), and/or very high triglycerides (e.g., at least about 500 mg/dl).

In another embodiment, the subject or subject group being treated has a baseline triglyceride level (or median baseline triglyceride level in the case of a subject group), fed or fasting, of at least about 200 mg/dl, at least about 300 mg/dl, at least about 400 mg/dl, at least about 500 mg/dl, at least about 600 mg/dl, at least about 700 mg/dl, at least about 800 mg/dl, at least about 900 mg/dl, at least about 1000 mg/dl, at least about 1100 mg/dl, at least about 1200 mg/dl, at least about 1300 mg/dl, at least about 1400 mg/dl, or at least about 1500 mg/dl, for example about 200 mg/dl to about 499 mg/dl, about 400 mg/dl to about 2500 mg/dl, about 450 mg/dl to about 2000 mg/dl or about 500 mg/dl to about 1500 mg/dl.

In one embodiment, the subject or subject group being treated in accordance with methods of the invention has previously been treated with Lovaza® and has experienced an increase in, or no decrease in, LDL-C levels and/or non-HDL-C levels. In one such embodiment, Lovaza® therapy is discontinued and replaced by a method of the present invention.

In another embodiment, the subject or subject group being treated in accordance with methods of the invention exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the invention exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2% or not more than about 0.15%. In one such embodiment, free plasma EPA and/or total fatty acid levels are determined prior to initiating therapy.

In another embodiment, the subject or subject group being treated in accordance with methods of the invention exhibits a fasting baseline absolute plasma level of total fatty acid (or mean thereof) not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the invention exhibits a fasting baseline plasma, serum or red blood cell membrane EPA level not greater than about 70 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present invention comprise a step of measuring the subject's (or subject group's mean) baseline lipid profile prior to initiating therapy. In another embodiment, methods of the invention comprise the step of identifying a subject or subject group having one or more of the following: baseline non-HDL-C value of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value of about 250 mg/dl to about 400 mg/dl, for example at least about 260 mg/dl, at least about 270 mg/dl, at least about 280 mg/dl or at least about 290 mg/dl; baseline vLDL-C value of about 140 mg/dl to about 200 mg/dl, for example at least about 150 mg/dl, at least about 160 mg/dl, at least about 170 mg/dl, at least about 180 mg/dl or at least about 190 mg/dl; baseline HDL-C value of about 10 to about 60 mg/dl, for example not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value of about 50 to about 300 mg/dl, for example not less than about 100 mg/dl, not less than about 90 mg/dl, not less than about 80 mg/dl, not less than about 70 mg/dl, not less than about 60 mg/dl or not less than about 50 mg/dl.

In another embodiment, the subject or subject group being treated has a baseline triglyceride level (or mean or median baseline triglyceride level in the case of a subject group), fed or fasting, of about 200 mg/dl to about 499 mg/dl. In another embodiment, the subject or subject group has a baseline LDL-C level (or mean or median baseline LDL-C level), despite stable statin therapy, of about 40 mg/dl to about 115 or about 40 to about 100 mg/dl.

In one embodiment, the subject or subject group being treated in accordance with methods of the invention is on concomitant statin therapy, for example atorvastatin, rosuvastatin or simvastatin therapy (with or without ezetimibe). In another embodiment, the subject is on concomitant stable statin therapy at time of initiation of ultra-pure EPA therapy.

In another embodiment, the subject or subject group being treated in accordance with methods of the invention has a body mass index (BMI or mean BMI) of not more than about 45 kg/m².

In one embodiment, the invention provides a method of lowering triglycerides in a subject on stable statin therapy having baseline fasting triglycerides of about 200 mg/dl to about 499 mg/dl, the method comprising administering to the subject a pharmaceutical composition comprising about 1 g to about 4 g of EPA or a derivative thereof, wherein upon administering the composition to the subject daily for a period of about 12 weeks the subject exhibits at least 10%, at least 15%, at least 20%, at least 25%, at least 30%, at least 35%, at least 40%, at least 45%, at least 50%, at least 55%, at least 60%, at least 65%, at least 70%, or at least 75% lower fasting triglycerides than a control subject maintained on stable statin therapy (and optionally placebo matching the ultra-pure EPA) without concomitant EPA for a period of about 12 weeks, wherein the control subject also has baseline fasting triglycerides of about 200 mg/dl to about 499 mg/dl. The term “stable statin therapy” herein means that the subject, subject group, control subject or control subject group in question has been taking a stable daily dose of a statin (e.g., atorvastatin, rosuvastatin or simvastatin) for at least 4 weeks prior to the baseline fasting triglyceride measurement (the “qualifying period”). For example, a subject or control subject on stable statin therapy would receive a constant daily (i.e. the same dose each day) statin dose for at least 4 weeks immediately prior to baseline fasting triglyceride measurement. In one embodiment, the subject's and control subject's LDL-C is maintained between about 40 mg/dl and about 115 mg/dl or about 40 mg/dl to about 100 mg/dl during the qualifying period. The subject and control subject are then continued on their stable statin dose for the 12 week period post baseline.

In one embodiment, the statin is administered to the subject and the control subject in an amount of about 1 mg to about 500 mg, about 5 mg to about 200 mg, or about 10 mg to about 100 mg, for example about 1 mg, about 2 mg, about 3 mg, about 4 mg, about 5 mg, about 6 mg, about 7 mg, about 8 mg, about 9 mg, or about 10 mg; about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In another embodiment, the subject (and optionally the control subject) has a baseline LDL-C level, despite stable statin therapy, of about 40 mg/dl to about 115 mg/dl or about 40 mg/dl to about 100 mg/dl. In another embodiment, the subject and/or control subject has a body mass index (BMI; or mean BMI) of not more than about 45 kg/m².

In another embodiment, the invention provides a method of lowering triglycerides in a subject group on stable statin therapy having mean baseline fasting triglycerides of about 200 mg/dl to about 499 mg/dl, the method comprising administering to members of the subject group a pharmaceutical composition comprising about 1 g to about 4 g of EPA per day, wherein upon administering the composition to the members of the subject group daily for a period of about 12 weeks the subject group exhibits at least 10%, at least 15%, at least 20%, at least 25%, at least 30%, at least 35%, at least 40%, at least 45%, at least 50%, at least 55%, at least 60%, at least 65%, at least 70%, at least 75% lower mean fasting triglycerides than a control subject group maintained on stable statin therapy without concomitant EPA (optionally with matching placebo) for a period of about 12 weeks, wherein the control subject group also has mean baseline fasting triglycerides of about 200 mg/dl to about 499 mg/dl. In a related embodiment, the stable statin therapy will be sufficient such that the subject group has a mean LDL-C level about at least about 40 mg/dl and not more than about 100 mg/dl or about 40 mg/dl to about 100 mg/dl for the 4 weeks immediately prior to the baseline fasting triglyceride measurement.

In another embodiment, the invention provides a method of lowering triglycerides in subject group on stable statin therapy and having a mean baseline fasting triglyceride level of about 200 mg/dl to about 499 mg/dl, the method comprising administering to members of the subject group a pharmaceutical composition comprising about 1 g to about 4 g of EPA, wherein upon administering the composition to members of the subject group daily for a period of about 12 weeks the subject group exhibits: (a) at least 10%, at least 15%, at least 20%, at least 25%, at least 30%, at least 35%, at least 40%, at least 45%, at least 50%, at least 55%, at least 60%, at least 65%, at least 70%, at least 75% lower mean fasting triglycerides by comparison with a control subject group maintained on stable statin therapy without concomitant EPA (optionally with matching placebo) for a period of about 12 weeks, and (b) no serum LDL-C increase, no statistically significant serum LDL-C increase, a serum LDL-C decrease, or the subject is statistically non-inferior to the control subjects (statin plus optional placebo) in regard to serum LDL-C elevation) no increase in mean serum LDL-C levels compared to baseline, wherein the control subject also has mean baseline fasting triglycerides of about 200 mg/dl to about 499 mg/dl.

In another embodiment, the invention provides a method of lowering triglycerides in subject on stable statin therapy and having mean baseline fasting triglyceride level of about 200 mg/dl to about 499 mg/dl, the method comprising administering to the subject a pharmaceutical composition comprising about 1 g to about 4 g of EPA, wherein upon administering the composition to the subject daily for a period of about 12 weeks the subject exhibits (a) at least 10%, at least 15%, at least 20%, at least 25%, at least 30%, at least 35%, at least 40%, at least 45%, at least 50%, at least 55%, at least 60%, at least 65%, at least 70%, or at least 75% lower fasting triglycerides by comparison with a control subject maintained on stable statin therapy without concomitant EPA for a period of about 12 weeks and (b) no increase in serum LDL-C levels compared to baseline, wherein the control subject also has baseline fasting triglycerides of about 200 mg/dl to about 499 mg/dl.

In another embodiment, the invention provides a method of lowering triglycerides in subject group on stable statin therapy and having mean baseline fasting triglyceride level of about 200 mg/dl to about 499 mg/dl, the method comprising administering to members of the subject group a pharmaceutical composition comprising about 1 g to about 4 g of EPA, wherein upon administering the composition to the members of the subject group daily for a period of about 12 weeks the subject group exhibits: (a) at least 10%, at least 15%, at least 20%, at least 25%, at least 30%, at least 35%, at least 40%, at least 45%, at least 50%, at least 55%, at least 60%, at least 65%, at least 70%, at least 75% lower mean fasting triglycerides and (b) at least 5%, at least 10%, at least 15%, at least 20%, at least 25%, at least 30%, at least 35%, at least 40%, at least 45% or at least 50% lower mean serum LDL-C levels by comparison with a control subject group maintained on stable statin therapy without concomitant EPA (optionally with matching placebo) for a period of about 12 weeks, no serum LDL-C increase, no statistically significant serum LDL-C increase, no statistically significant serum LDL-C increase, a serum LDL-C decrease, or the subject group is statistically non-inferior to the control subject group (statin plus optional placebo) in regard to serum LDL-C elevation), wherein the control subject group also has mean baseline fasting triglycerides of about 200 mg/dl to about 499 mg/dl.

In another embodiment, the invention provides a method of lowering triglycerides in subject group on stable statin therapy and having mean baseline fasting triglyceride level of about 200 mg/dl to about 499 mg/dl, the method comprising administering to members of the subject group a pharmaceutical composition comprising about 1 g to about 4 g of EPA, wherein upon administering the composition to the members of the subject group daily for a period of about 12 weeks the subject group exhibits (a) at least 10%, at least 15%, at least 20%, at least 25%, at least 30%, at least 35%, at least 40%, at least 45%, at least 50%, at least 55%, at least 60%, at least 65%, at least 70%, at least 75% lower mean fasting triglycerides and (b) at least 5%, at least 10%, at least 15%, at least 20%, at least 25%, at least 30%, at least 35%, at least 40%, at least 45% or at least 50% lower mean serum LDL-C levels by comparison with a control subject group maintained on stable statin therapy without concomitant EPA (optionally with matching placebo) for a period of about 12 weeks, no serum LDL-C increase, no statistically significant serum LDL-C increase, no statistically significant serum LDL-C increase, a serum LDL-C decrease, or the subject group is statistically non-inferior to the control subject group (statin plus optional placebo) in regard to serum LDL-C elevation), wherein the control subject group also has mean baseline fasting triglycerides of about 200 mg/dl to about 499 mg/dl.

In another embodiment, the subject or subject group being treated in accordance with methods of the invention exhibits a fasting baseline absolute plasma level of free total fatty acid (or mean thereof) not greater than about 300 nmol/ml, not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the invention exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the invention exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2% or not more than about 0.15%. In one such embodiment, free plasma EPA and/or total fatty acid levels are determined prior to initiating therapy.

In another embodiment, the subject or subject group being treated in accordance with methods of the invention exhibits a fasting baseline absolute plasma level of free EPA (or mean thereof) not greater than about 1 nmol/ml, not greater than about 0.75 nmol/ml, not greater than about 0.50 nmol/ml, not greater than about 0.4 nmol/ml, not greater than about 0.35 nmol/ml, or not greater than about 0.30 nmol/ml.

In another embodiment, the subject or subject group being treated in accordance with methods of the invention exhibits a fasting baseline plasma, serum or red blood cell membrane EPA level not greater than about 150 μg/ml, not greater than about 125 μg/ml, not greater than about 100 μg/ml, not greater than about 95 μg/ml, not greater than about 75 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present invention comprise a step of measuring the subject's (or subject group's mean) baseline lipid profile prior to initiating therapy. In another embodiment, methods of the invention comprise the step of identifying a subject or subject group having one or more of the following: baseline non-HDL-C value (or mean) of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value (or mean) of about 250 mg/dl to about 400 mg/dl, for example at least about 260 mg/dl, at least about 270 mg/dl, at least about 280 mg/dl or at least about 290 mg/dl; baseline vLDL-C value (or mean) of about 140 mg/dl to about 200 mg/dl, for example at least about 150 mg/dl, at least about 160 mg/dl, at least about 170 mg/dl, at least about 180 mg/dl or at least about 190 mg/dl; baseline HDL-C value (or mean) of about 10 to about 100 mg/dl, for example not more than about 90 mg/dl not, not more than about 80 mg/di, not more than about 70 mg/di, not more than about 60 mg/di, not more than about 60 mg/di, not more than about 50 mg/di, not more than about 40 mg/di, not more than about 35 mg/di, not more than about 30 mg/di, not more than about 25 mg/di, not more than about 20 mg/di, or not more than about 15 mg/di; and/or baseline LDL-C value (or mean) of about 30 to about 300 mg/dl, for example not less than about 40 mg/dl, not less than about 50 mg/dl, not less than about 60 mg/dl, not less than about 70 mg/dl, not less than about 90 mg/dl or not less than about 90 mg/dl.

In a related embodiment, upon treatment in accordance with the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits one or more of the following outcomes:

(a) reduced triglyceride levels compared to baseline;

(b) reduced Apo B levels compared to baseline;

(c) increased HDL-C levels compared to baseline;

(d) no increase in LDL-C levels compared to baseline;

(e) a reduction in LDL-C levels compared to baseline;

(f) a reduction in non-HDL-C levels compared to baseline;

(g) a reduction in vLDL levels compared to baseline;

(h) an increase in apo A-I levels compared to baseline;

(i) an increase in apo A-I/apo B ratio compared to baseline;

(j) a reduction in lipoprotein A levels compared to baseline;

(k) a reduction in LDL particle number compared to baseline;

(l) an increase in LDL size compared to baseline;

(m) a reduction in remnant-like particle cholesterol compared to baseline;

(n) a reduction in oxidized LDL compared to baseline;

(o) no change or a reduction in fasting plasma glucose (FPG) compared to baseline;

(p) a reduction in hemoglobin A_{1c} (HbA_{1c}) compared to baseline;

(q) a reduction in homeostasis model insulin resistance compared to baseline;

(r) a reduction in lipoprotein associated phospholipase A2 compared to baseline;

(s) a reduction in intracellular adhesion molecule-1 compared to baseline;

(t) a reduction in interleukin-6 compared to baseline;

(u) a reduction in plasminogen activator inhibitor-1 compared to baseline;

(v) a reduction in high sensitivity C-reactive protein (hsCRP) compared to baseline;

(w) an increase in serum or plasma EPA compared to baseline;

(x) an increase in red blood cell (RBC) membrane EPA compared to baseline; and/or

(y) a reduction or increase in one or more of serum phospholipid and/or red blood cell content of docosahexaenoic acid (DHA), docosapentaenoic acid (DPA), arachidonic acid (AA), palmitic acid (PA), staeridonic acid (SA) or oleic acid (OA) compared to baseline.

In one embodiment, upon administering a composition of the invention to a subject, the subject exhibits a decrease in triglyceride levels, an increase in the concentrations of EPA and DPA (n−3) in red blood cells, and an increase of the ratio of EPA:arachidonic acid in red blood cells. In a related embodiment the subject exhibits substantially no or no increase in RBC DHA.

In one embodiment, methods of the present invention comprise measuring baseline levels of one or more markers set forth in (a)-(y) above prior to dosing the subject or subject group. In another embodiment, the methods comprise administering a composition as disclosed herein to the subject after baseline levels of one or more markers set forth in (a)-(y) are determined, and subsequently taking an additional measurement of said one or more markers.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, or all 25 of outcomes (a)-(y) described immediately above.

In another embodiment, upon treatment with a composition of the present invention, the subject or subject group exhibits one or more of the following outcomes:

(a) a reduction in triglyceride level of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(b) a less than 30% increase, less than 20% increase, less than 10% increase, less than 5% increase or no increase in non-HDL-C levels or a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(c) substantially no change in HDL-C levels, no change in HDL-C levels, or an increase in HDL-C levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(d) a less than 60% increase, a less than 50% increase, a less than 40% increase, a less than 30% increase, less than 20% increase, less than 10% increase, less than 5% increase or no increase in LDL-C levels or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55% or at least about 75% (actual % change or median % change) as compared to baseline;

(e) a decrease in Apo B levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(f) a reduction in vLDL levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(g) an increase in apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(h) an increase in apo A-I/apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(i) a reduction in lipoprotein (a) levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(j) a reduction in mean LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(k) an increase in mean LDL particle size of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(l) a reduction in remnant-like particle cholesterol of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(m) a reduction in oxidized LDL of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(n) substantially no change, no significant change, or a reduction (e.g., in the case of a diabetic subject) in fasting plasma glucose (FPG) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(o) substantially no change, no significant change or a reduction in hemoglobin A_{1c} (HbA_{1c}) of at least about 5%, at least about 10%, at least about 15%, at least about 20%, at least about 25%, at least about 30%, at least about 35%, at least about 40%, at least about 45%, or at least about 50% (actual % change or median % change) compared to baseline;

(p) a reduction in homeostasis model index insulin resistance of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(q) a reduction in lipoprotein associated phospholipase A2 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(r) a reduction in intracellular adhesion molecule-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(s) a reduction in interleukin-6 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(t) a reduction in plasminogen activator inhibitor-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(u) a reduction in high sensitivity C-reactive protein (hsCRP) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(v) an increase in serum, plasma and/or RBC EPA of at least about 5%, at least about 10%, at least about 15%, at least about 20%, at least about 25%, at least about 30%, at least about 35%, at least about 40%, at least about 45%, at least about 50%, at least about 100%, at least about 200% or at least about 400% (actual % change or median % change) compared to baseline;

(w) an increase in serum phospholipid and/or red blood cell membrane EPA of at least about 5%, at least about 10%, at least about 15%, at least about 20%, at least about 25%, at least about 30%, at least about 35%, at least about 40%, at least about 45%, at least about 50%, at least about 100%, at least about 200%, or at least about 400% (actual % change or median % change) compared to baseline;

(x) a reduction or increase in one or more of serum phospholipid and/or red blood cell DHA, DPA, AA, PA and/or OA of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline; and/or

(y) a reduction in total cholesterol of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline.

In one embodiment, methods of the present invention comprise measuring baseline levels of one or more markers set forth in (a)-(y) prior to dosing the subject or subject group. In another embodiment, the methods comprise administering a composition as disclosed herein to the subject after baseline levels of one or more markers set forth in (a)-(y) are determined, and subsequently taking a second measurement of the one or more markers as measured at baseline for comparison thereto.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, or all 25 of outcomes (a)-(y) described immediately above.

Parameters (a)-(y) can be measured in accordance with any clinically acceptable methodology. For example, triglycerides, total cholesterol, HDL-C and fasting blood sugar can be sample from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A2 can be determined from EDTA plasma or serum and serum, respectively, using enzymatic immunoseparation techniques. Oxidized LDL, intercellular adhesion molecule-1 and interleukin-6 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company.

In one embodiment, subjects fast for up to 12 hours prior to blood sample collection, for example about 10 hours.

In another embodiment, the present invention provides a method of treating or preventing primary hypercholesterolemia, borderline high/high triglycerides (e.g., about 200 mg/dl to about 499 mg/dl), very high triglycerides (e.g., at least about 500 mg/dl), and/or mixed dyslipidemia (Fredrickson Types IIa and IIb) in a patient in need thereof, comprising administering to the patient one or more compositions as disclosed herein. In a related embodiment, the present invention provides a method of reducing triglyceride levels in a subject or subjects when treatment with a statin or niacin extended-release monotherapy is considered inadequate (Frederickson type IV hyperlipidemia).

In another embodiment, the present invention provides a method of treating or preventing risk of recurrent nonfatal myocardial infarction in a patient with a history of myocardial infarction, comprising administering to the patient one or more compositions as disclosed herein.

In another embodiment, the present invention provides a method of slowing progression of or promoting regression of atherosclerotic disease in a patient in need thereof, comprising administering to a subject in need thereof one or more compositions as disclosed herein.

In another embodiment, the present invention provides a method of treating or preventing very high serum triglyceride levels (e.g., Types IV and V hyperlipidemia) in a patient in need thereof, comprising administering to the patient one or more compositions as disclosed herein.

In another embodiment, the present invention provides a method of treating subjects having very high serum triglyceride levels (e.g., greater than 1000 mg/dl or greater than 2000 mg/dl) and that are at risk of developing pancreatitis, comprising administering to the patient one or more compositions as disclosed herein.

In one embodiment, a composition or dosage unit of the invention or suitable for use in methods disclosed herein comprises or provides a daily dose of eicosapentaenoic acid of about 1 mg to about 10,000 mg, 25 about 5000 mg, about 50 to about 4000 mg, about 75 mg to about 3000 mg, or about 100 mg to about 1000 mg, for example about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5050 mg, about 5100 mg, about 5150 mg, about 5200 mg, about 5250 mg, about 5300 mg, about 5350 mg, about 5400 mg, about 5450 mg, about 5500 mg, about 5550 mg, about 5600 mg, about 5650 mg, about 5700 mg, about 5750 mg, about 5800 mg, about 5850 mg, about 5900 mg, about 5950 mg, about 6000 mg, about 6050 mg, about 6100 mg, about 6150 mg, about 6200 mg, about 6250 mg, about 6300 mg, about 6350 mg, about 6400 mg, about 6450 mg, about 6500 mg, about 6550 mg, about 6600 mg, about 6650 mg, about 6700 mg, about 6750 mg, about 6800 mg, about 6850 mg, about 6900 mg, about 6950 mg, about 7000 mg, about 7050 mg, about 7100 mg, about 7150 mg, about 7200 mg, about 7250 mg, about 7300 mg, about 7350 mg, about 7400 mg, about 7450 mg, about 7500 mg, about 7550 mg, about 7600 mg, about 7650 mg, about 7700 mg, about 7750 mg, about 7800 mg, about 7850 mg, about 7900 mg, about 7950 mg, about 8000 mg, about 8050 mg, about 8100 mg, about 8150 mg, about 8200 mg, about 8250 mg, about 8300 mg, about 8350 mg, about 8400 mg, about 8450 mg, about 8500 mg, about 8550 mg, about 8600 mg, about 8650 mg, about 8700 mg, about 8750 mg, about 8800 mg, about 8850 mg, about 8900 mg, about 8950 mg, about 9000 mg, about 9050 mg, about 9100 mg, about 9150 mg, about 9200 mg, about 9250 mg, about 9300 mg, about 9350 mg, about 9400 mg, about 9450 mg, about 9500 mg, about 9550 mg, about 9600 mg, about 9650 mg, about 9700 mg, about 9750 mg, about 9800 mg, about 9850 mg, about 9900 mg, about 9950 mg, or about 10,000 mg.

In another embodiment, any of the methods disclosed herein are used in treatment or prevention of a subject or subjects that consume a traditional Western diet. In one embodiment, the methods of the invention include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60% or more or 70% of total calories come from these sources.

In another embodiment, a composition useful in accordance with the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight, docosahexaenoic acid (DHA), if any. In another embodiment, a composition of the invention contains substantially no docosahexaenoic acid. In still another embodiment, a composition useful in the present invention contains no docosahexaenoic acid and/or derivative thereof.

In another embodiment, a composition or dosage unit or suitable for use in methods of the invention contains less than 10%, less than 9%, less than 8%, less than 7%, less than 6%, less than 5%, less than 4%, less than 3%, less than 2%, less than 1%, less than 0.5% or less than 0.25%, by weight of the total composition or by weight of the total fatty acid content, of any fatty acid other than EPA. Illustrative examples of a “fatty acid other than EPA” include linolenic acid (LA), arachidonic acid (AA), docosahexaenoic acid (DHA), alpha-linolenic acid (ALA), stearadonic acid (STA), eicosatrienoic acid (ETA) and/or docosapentaenoic acid (DPA). In another embodiment, a composition useful in accordance with methods of the invention contains about 0.1% to about 4%, about 0.5% to about 3%, or about 1% to about 2%, by weight, of total fatty acids other than EPA and/or DHA.

In another embodiment, a composition or dosage unit or suitable for use in methods of the invention has one or more of the following features: (a) eicosapentaenoic acid ethyl ester represents at least about 96%, at least about 97%, or at least about 98%, by weight, of all fatty acids present in the composition; (b) the composition contains not more than about 4%, not more than about 3%, or not more than about 2%, by weight, of total fatty acids other than eicosapentaenoic acid ethyl ester; (c) the composition contains not more than about 0.6%, not more than about 0.5%, or not more than about 0.4% of any individual fatty acid other than eicosapentaenoic acid ethyl ester; (d) the composition has a refractive index (20° C.) of about 1 to about 2, about 1.2 to about 1.8 or about 1.4 to about 1.5; (e) the composition has a specific gravity (20° C.) of about 0.8 to about 1.0, about 0.85 to about 0.95 or about 0.9 to about 0.92; (e) the composition contains not more than about 20 ppm, not more than about 15 ppm or not more than about 10 ppm heavy metals, (f) the composition contains not more than about 5 ppm, not more than about 4 ppm, not more than about 3 ppm, or not more than about 2 ppm arsenic, and/or (g) the composition has a peroxide value of not more than about 5 meq/kg, not more than about 4 meq/kg, not more than about 3 meq/kg, or not more than about 2 meq/kg.

In another embodiment, a composition or dosage unit or suitable for use in methods of the invention comprises, consists of or consists essentially of at least 95% by weight ethyl eicosapentaenoate (EPA-E), about 0.2% to about 0.5% by weight ethyl octadecatetraenoate (ODTA-E), about 0.05% to about 0.25% by weight ethyl nonadecapentaenoate (NDPA-E), about 0.2% to about 0.45% by weight ethyl arachidonate (AA-E), about 0.3% to about 0.5% by weight ethyl eicosatetraenoate (ETA-E), and about 0.05% to about 0.32% ethyl heneicosapentaenoate (HPA-E). In another embodiment, the composition is present in a capsule shell.

In another embodiment, a composition or dosage unit or suitable for use in methods of the invention comprises, consists essentially of, or consists of at least 95%, 96% or 97%, by weight, ethyl eicosapentaenoate, about 0.2% to about 0.5% by weight ethyl octadecatetraenoate, about 0.05% to about 0.25% by weight ethyl nonadecapentaenoate, about 0.2% to about 0.45% by weight ethyl arachidonate, about 0.3% to about 0.5% by weight ethyl eicosatetraenoate, and about 0.05% to about 0.32% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g., tocopherol) or other impurities in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell.

In another embodiment, a composition or dosage unit or suitable for use in methods of the invention comprises, consists essentially of, or consists of at least 96% by weight ethyl eicosapentaenoate, about 0.22% to about 0.4% by weight ethyl octadecatetraenoate, about 0.075% to about 0.20% by weight ethyl nonadecapentaenoate, about 0.25% to about 0.40% by weight ethyl arachidonate, about 0.3% to about 0.4% by weight ethyl eicosatetraenoate and about 0.075% to about 0.25% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g., tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell. In one embodiment, the dosage form is a gel or liquid capsule and is packaged in blister packages of about 1 to about 20 capsules per sheet.

In another embodiment, a composition or dosage unit or suitable for use in methods of the invention comprises, consists essentially of, or consists of at least 96%, 97% or 98%, by weight, ethyl eicosapentaenoate, about 0.25% to about 0.38% by weight ethyl octadecatetraenoate, about 0.10% to about 0.15% by weight ethyl nonadecapentaenoate, about 0.25% to about 0.35% by weight ethyl arachidonate, about 0.31% to about 0.38% by weight ethyl eicosatetraenoate, and about 0.08% to about 0.20% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g., tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell.

In another embodiment, a composition or dosage unit or suitable for use in methods of the invention is administered to a subject once or twice per day. In another embodiment, 1, 2, 3 or 4 capsules, each containing about 900 mg to about 1.1 g (e.g., about 1 g) of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 pm and about 11 pm.

In one embodiment, a subject being treated in accordance with methods of the invention is not otherwise on lipid-altering therapy, for example statin, fibrate, niacin and/or ezetimibe therapy.

In another embodiment, a composition or dosage unit or suitable for use in methods of the invention is orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition or dosage unit or suitable for use in methods of the invention can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In another embodiment, the invention provides use of any composition described herein for treating moderate to severe hypertriglyceridemia in a subject in need thereof, comprising: providing a subject having a fasting baseline triglyceride level of about 500 mg/dl to about 1500 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid.

In one embodiment, compositions of the invention, upon storage in a closed container maintained at room temperature, refrigerated (e.g., about 5 to about 5-10° C.) temperature, or frozen for a period of about 1, 2, 3, 4, 5, 6, 7, 8, 9, 10, 11, or 12 months, exhibit at least about 90%, at least about 95%, at least about 97.5%, or at least about 99% of the active ingredient(s) originally present therein.

In one embodiment, the invention provides use of a composition as described herein in manufacture of a medicament for treatment of any of a cardiovascular-related disease. In another embodiment, the subject is diabetic.

In one embodiment, a composition as set forth herein is packaged together with instructions for using the composition to treat a cardiovascular disorder.

It will be appreciated that the pharmacological activity of the compositions of the invention can be demonstrated using standard pharmacological models that are known in the art. Furthermore, it will be appreciated that the disclosed compositions can be incorporated or encapsulated in a suitable polymer matrix or membrane for site-specific delivery, or can be functionalized with specific targeting agents capable of effecting site specific delivery. These techniques, as well as other drug delivery techniques, are well known in the art.

In one embodiment, a pharmaceutical composition of the present disclosure provides a mean total eicosapentaenoic acid, unesterified plasma eicosapentaenoic acid, or total eicosapentaenoic acid red blood cell Cₘₐₓ of about 0.5 μg/mL to about 500 μg/mL, for example about 105 μg/mL to about 204 μg/mL, about 235 μg/mL to about 460 μg/mL, about 0.5 μg/mL, about 0.6 μg/mL, about 0.7 μg/mL (e.g, about 0.66 μg/mL), about 1 μg/mL, about 1.4 μg/mL, about 5 μg/mL, about 10 μg/mL, about 15 μg/mL, about 20 μg/mL, about 25 μg/mL, about 30 μg/mL, about 31 μg/mL, about 35 μg/mL, about 38 μg/mL (e.g, about 37.6 μg/mL), about 40 μg/mL, about 42 μg/mL (e.g, about 42.3 μg/mL), about 45 μg/mL, about 50 μg/mL, about 55 μg/mL, about 60 μg/mL, about 65 μg/mL, about 70 μg/mL, about 75 μg/mL, about 77 μg/mL (e.g, about 76.7 μg/mL), about 80 μg/mL, about 85 μg/mL, about 90 μg/mL, about 95 μg/mL, about 100 μg/mL, about 105 μg/mL, about 110 μg/mL, about 115 μg/mL, about 120 μg/mL, about 125 μg/mL, about 130 μg/mL, about 135 μg/mL, about 140 μg/mL, about 145 μg/mL, about 150 μg/mL, about 155 μg/mL (e.g, about 154.9 μg/mL), about 160 μg/mL, about 165 μg/mL, about 170 μg/mL, about 175 μg/mL, about 180 μg/mL, about 185 μg/mL, about 190 μg/mL, about 195 μg/mL, about 200 μg/mL, about 205 μg/mL, about 210 μg/mL (e.g, about 210.5 μg/mL), about 215 μg/mL, about 220 μg/mL, about 225 μg/mL, about 230 μg/mL, about 233 μg/mL (e.g, about 232.8 μg/mL), about 235 μg/mL, about 240 μg/mL, about 245 μg/mL, about 250 μg/mL, about 255 μg/mL, about 260 μg/mL, about 265 μg/mL, about 270 μg/mL, about 275 μg/mL, about 280 μg/mL, about 285 μg/mL, about 290 μg/mL, about 295 μg/mL, about 300 μg/mL, about 305 μg/mL, about 310 μg/mL, about 315 μg/mL, about 320 μg/mL, about 325 μg/mL, about 330 μg/mL, about 335 μg/mL, about 340 μg/mL, about 345 μg/mL, about 347 μg/mL (e.g., about 347.2 μg/mL), about 350 μg/mL, about 355 μg/mL, about 360 μg/mL, about 365 μg/mL, about 370 μg/mL, about 375 μg/mL, about 380 μg/mL, about 385 μg/mL, about 390 μg/mL, about 395 μg/mL, about 400 μg/mL, about 405 μg/mL, about 410 μg/mL, about 415 μg/mL, about 420 μg/mL, about 425 μg/mL, about 430 μg/mL, about 435 μg/mL, about 440 μg/mL, about 445 μg/mL, about 450 μg/mL, about 455 μg/mL, about 460 μg/mL, about 465 μg/mL, about 470 μg/mL, about 475 μg/mL, about 480 μg/mL, about 485 μg/mL, about 490 μg/mL, about 495 μg/mL, or about 500 μg/mL.

In one embodiment, a pharmaceutical composition of the present disclosure provides a mean total eicosapentaenoic acid, unesterified plasma eicosapentaenoic acid, or total eicosapentaenoic acid red blood cell AUC₀₋₂₄ₕ of about 1 μg·h/mL to about 7000 μg·h/mL, for example about 1 μg·h/mL to about 20 μg·h/mL, about 500 μg·h/mL to about 1500 μg·h/mL, about 2500 μg·h/mL to about 7000 μg·h/mL, about 1 μg·h/mL, about 2 μg·h/mL, about 3 μg·h/mL, about 4 μg·h/mL, about 5 μg·h/mL, about 6 μg·h/mL, about 7 μg·h/mL, about 8 μg·h/mL, about 9 μg·h/mL, about 10 μg·h/mL, about 11 μg·h/mL, about 12 μg·h/mL, about 13 μg·h/mL, about 14 μg·h/mL, about 15 μg·h/mL, about 16 μg·h/mL, about 17 μg·h/mL, about 18 μg·h/mL, about 19 μg·h/mL, about 20 μg·h/mL, about 500 μg·h/mL, about 550 μg·h/mL, about 600 μg·h/mL, about 650 μg·h/mL, about 700 μg·h/mL, about 750 μg·h/mL, about 800 μg·h/mL, about 850 μg·h/mL, about 900 μg·h/mL, about 950 μg·h/mL, about 1000 μg·h/mL, about 1050 μg·h/mL, about 1100 μg·h/mL, about 1150 μg·h/mL, about 1200 μg·h/mL, about 1250 μg·h/mL, about 1300 μg·h/mL, about 1350 μg·h/mL, about 1400 μg·h/mL, about 1450 μg·h/mL, about 1500 μg·h/mL, about 2500 μg·h/mL, about 2550 μg·h/mL, about 2600 μg·h/mL, about 2650 μg·h/mL, about 2700 μg·h/mL, about 2750 μg·h/mL, about 2800 μg·h/mL, about 2850 μg·h/mL, about 2900 μg·h/mL, about 2950 μg·h/mL, about 3000 μg·h/mL, about 3050 μg·h/mL, about 3100 μg·h/mL, about 3150 μg·h/mL, about 3200 μg·h/mL, about 3250 μg·h/mL, about 3300 μg·h/mL, about 3350 μg·h/mL, about 3400 μg·h/mL, about 3450 μg·h/mL, about 3500 μg·h/mL, about 3550 μg·h/mL, about 3600 μg·h/mL, about 3650 μg·h/mL, about 3700 μg·h/mL, about 3750 μg·h/mL, about 3800 μg·h/mL, about 3850 μg·h/mL, about 3900 μg·h/mL, about 3950 μg·h/mL, about 4000 μg·h/mL, about 4050 μg·h/mL, about 4100 μg·h/mL, about 4150 μg·h/mL, about 4200 μg·h/mL, about 4250 μg·h/mL, about 4300 μg·h/mL, about 4350 μg·h/mL, about 4400 μg·h/mL, about 4450 μg·h/mL, about 4500 μg·h/mL, about 4550 μg·h/mL, about 4600 μg·h/mL, about 4650 μg·h/mL, about 4700 μg·h/mL, about 4750 μg·h/mL, about 4800 μg·h/mL, about 4850 μg·h/mL, about 4900 μg·h/mL, about 4950 μg·h/mL, about 5000 μg·h/mL, about 5050 μg·h/mL, about 5100 μg·h/mL, about 5150 μg·h/mL, about 5200 μg·h/mL, about 5250 μg·h/mL, about 5300 μg·h/mL, about 5350 μg·h/mL, about 5400 μg·h/mL, about 5450 μg·h/mL, about 5500 μg·h/mL, about 5550 μg·h/mL, about 5600 μg·h/mL, about 5650 μg·h/mL, about 5700 μg·h/mL, about 5750 μg·h/mL, about 5800 μg·h/mL, about 5850 μg·h/mL, about 5900 μg·h/mL, about 5950 μg·h/mL, about 6000 μg·h/mL, about 6050 μg·h/mL, about 6100 μg·h/mL, about 6150 μg·h/mL, about 6200 μg·h/mL, about 6250 μg·h/mL, about 6300 μg·h/mL, about 6350 μg·h/mL, about 6400 μg·h/mL, about 6450 μg·h/mL, about 6500 μg·h/mL, about 6550 μg·h/mL, about 6600 μg·h/mL, about 6650 μg·h/mL, about 6700 μg·h/mL, about 6750 μg·h/mL, about 6800 μg·h/mL, about 6850 μg·h/mL, about 6900 μg·h/mL, about 6950 μg·h/mL, or about 7000 μg·h/mL.

In one embodiment, a pharmaceutical composition of the present disclosure provides a mean total eicosapentaenoic acid, unesterified plasma eicosapentaenoic acid, or total eicosapentaenoic acid red blood cell Cₘᵢₙ of about 0.2 μg/mL to about 250 μg/mL, for example about 0.2 μg/mL to about 2 μg/mL, about 20 μg/mL to about 70 μg/mL, about 70 μg/mL to about 215 μg/mL, about 0.2 μg/mL, about 0.3 μg/mL, about 0.4 μg/mL (e.g., about 0.36 μg/mL, about 0.41 μg/mL or about 0.44 μg/mL), about 0.5 μg/mL, about 0.6 μg/mL, about 0.7 μg/mL, about 0.8 μg/mL, about 0.9 μg/mL, about 1 μg/mL (e.g., about 1.06 μg/mL), about 1.1 μg/mL, about 1.2 μg/mL, about 1.3 μg/mL, about 1.4 μg/mL, about 1.5 μg/mL, about 1.6 μg/mL, about 1.7 μg/mL, about 1.8 μg/mL, about 1.9 μg/mL, about 2 μg/mL, about 5 μg/mL, about 10 μg/mL, about 15 μg/mL, about 18 μg/mL, about 20 μg/mL, about 21 μg/mL (e.g., about 20.7 μg/mL), about 25 μg/mL, about 26 μg/mL (e.g., about 26.3 μg/mL), about 30 μg/mL, about 31 μg/mL (e.g., about 31.2 μg/mL), about 35 μg/mL, about 40 μg/mL, about 45 μg/mL, about 50 μg/mL, about 55 μg/mL, about 60 μg/mL, about 64 μg/mL (e.g., about 64.1 μg/mL), about 65 μg/mL, about 70 μg/mL (e.g., about 69.7 μg/mL), about 75 μg/mL (e.g., about 75.1 μg/mL), about 80 μg/mL, about 84 μg/mL (e.g., about 83.5 μg/mL), about 85 μg/mL, about 89 μg/mL (e.g., about 89.3 μg/mL), about 90 μg/mL, about 95 μg/mL, about 100 μg/mL, about 101 μg/mL, about 104 μg/mL, about 105 μg/mL, about 110 μg/mL, about 115 μg/mL, about 120 μg/mL, about 125 μg/mL, about 130 μg/mL, about 135 μg/mL, about 140 μg/mL, about 145 μg/mL, about 150 μg/mL, about 155 μg/mL, about 160 μg/mL, about 165 μg/mL, about 170 μg/mL, about 175 μg/mL, about 180 μg/mL, about 185 μg/mL, about 190 μg/mL, about 195 μg/mL, about 200 μg/mL, about 205 μg/mL, about 210 μg/mL, about 215 μg/mL, about 220 μg/mL, about 225 μg/mL, about 230 μg/mL, about 235 μg/mL, about 240 μg/mL, about 245 μg/mL, or about 250 μg/mL.

In one embodiment, a pharmaceutical composition of the present disclosure provides a mean total eicosapentaenoic acid, unesterified plasma eicosapentaenoic acid, or total eicosapentaenoic acid red blood cell Tₘₐₓ of about 1 hour to about 36 hours, for example about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours.

In one embodiment, a pharmaceutical composition of the present disclosure provides a mean total eicosapentaenoic acid, unesterified plasma eicosapentaenoic acid, or total eicosapentaenoic acid red blood cell T_{1/2} of about 50 hours to about 700 hours, for example about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 135 hours, about 140 hours, about 145 hours, about 150 hours, about 155 hours, about 160 hours, about 165 hours, about 170 hours, about 175 hours, about 180 hours, about 185 hours, about 190 hours, about 195 hours, about 200 hours, about 205 hours, about 210 hours, about 215 hours, about 220 hours, about 225 hours, about 230 hours, about 235 hours, about 240 hours, about 245 hours, about 250 hours, about 255 hours, about 260 hours, about 265 hours, about 270 hours, about 275 hours, about 280 hours, about 285 hours, about 290 hours, about 295 hours, about 300 hours, about 305 hours, about 310 hours, about 315 hours, about 320 hours, about 325 hours, about 330 hours, about 335 hours, about 340 hours, about 345 hours, about 350 hours, about 355 hours, about 360 hours, about 365 hours, about 370 hours, about 375 hours, about 380 hours, about 385 hours, about 390 hours, about 395 hours, about 400 hours, about 405 hours, about 410 hours, about 415 hours, about 420 hours, about 425 hours, about 430 hours, about 435 hours, about 440 hours, about 445 hours, about 450 hours, about 455 hours, about 460 hours, about 465 hours, about 470 hours, about 475 hours, about 480 hours, about 485 hours, about 490 hours, about 495 hours, about 500 hours, about 505 hours, about 510 hours, about 515 hours, about 520 hours, about 525 hours, about 530 hours, about 535 hours, about 540 hours, about 545 hours, about 550 hours, about 555 hours, about 560 hours, about 565 hours, about 570 hours, about 575 hours, about 580 hours, about 585 hours, about 590 hours, about 595 hours, about 600 hours, about 605 hours, about 610 hours, about 615 hours, about 620 hours, about 625 hours, about 630 hours, about 635 hours, about 640 hours, about 645 hours, about 650 hours, about 655 hours, about 660 hours, about 665 hours, about 670 hours, about 675 hours, about 680 hours, about 685 hours, about 690 hours, about 695 hours, or about 700 hours.

In one embodiment, a pharmaceutical composition of the present disclosure provides a mean total eicosapentaenoic acid, unesterified plasma eicosapentaenoic acid, or total eicosapentaenoic acid red blood cell CL/F of about 500 mL/hr to about 400,000 mL/hr, for example about 500 mL/hr, about 525 mL/hr, about 550 mL/hr, about 575 mL/hr, about 600 mL/hr, about 625 mL/hr, about 650 mL/hr, about 675 mL/hr, about 684 mL/hr (e.g., about 683.7 mL/hr), about 658 mL/hr (e.g., about 684.5 mL/hr), about 700 mL/hr, about 725 mL/hr, about 750 mL/hr, about 775 mL/hr, about 776 mL/hr (e.g., about 776.4 mL/hr), about 800 mL/hr, about 825 mL/hr, about 850 mL/hr, about 868 mL/hr (e.g., about 867.7 mL/hr), about 875 mL/hr, about 900 mL/hr, about 925 mL/hr, about 950 mL/hr, about 975 mL/hr, about 1,000 mL/hr, about 200,000 mL/hr, about 210,000 mL/hr, about 220,000 mL/hr, about 230,000 mL/hr, about 240,000 mL/hr, about 250,000 mL/hr, about 260,000 mL/hr, about 270,000 mL/hr, about 280,000 mL/hr, about 290,000 mL/hr, about 300,000 mL/hr, about 310,000 mL/hr, about 320,000 mL/hr, about 330,000 mL/hr, about 340,000 mL/hr, about 350,000 mL/hr, about 360,000 mL/hr, about 370,000 mL/hr, about 380,000 mL/hr, about 390,000 mL/hr, or about 400,000 mL/hr.

In one embodiment, a pharmaceutical composition of the present disclosure provides a mean total eicosapentaenoic acid, unesterified plasma eicosapentaenoic acid, or total eicosapentaenoic acid red blood cell V_{z}/F of about 50 L to about 50,000 L, for example about 75 L to about 90 L, about 28,000 L to about 41,000 L, about 50 L, about 55 L, about 60 L, about 65 L, about 70 L, about 75 L, about 79 L (e.g., about 79.3 L), about 80 L (e.g., about 79.8 L), about 85 L, about 88 L (e.g., about 88.4 L), about 90 L, about 95 L, about 100 L, about 28,000 L, about 28,200 L, about 29,000 L, about 30,000 L, about 31,000 L, about 32,000 L, about 33,000 L, about 33,160 L (e.g., about 33,164 L), about 34,000 L, about 35,000 L, about 36,000 L, about 37,000 L, about 38,000 L, about 39,000 L, about 40,000 L, about 40,870 L (e.g., about 40,873 L), about 41,000 L, about 42,000 L, about 43,000 L, about 44,000 L, about 45,000 L, about 46,000 L, about 47,000 L, about 48,000 L, about 49,000 L, or about 50,000 L.

In one embodiment, a pharmaceutical composition of the present disclosure comprising about 1 g of ethyl eicosapentaenoate (e.g., about 800 mg to about 1200 mg, about 850 mg to about 1150 mg, about 900 mg to about 1100 mg, about 950 mg to about 1050 mg, or about 975 mg to about 1025 mg) provides a mean plasma Cₘₐₓ of about 154.9+/−49.4 μg/mL, a mean plasma AUC₀₋₂₄ₕ of about 2907+/−1160 μg·h/mL, a median plasma Tₘₐₓ of about 5 hours, and a mean plasma T_{1/2} of about 75.1+/−46.5 hours of ethyl eicosapentaenoate when administered orally twice per day to a human subject. In some embodiments, the pharmaceutical composition further provides a mean plasma Cₘᵢₙ of about 101.0+/−50.4 μg/mL of ethyl eicosapentaenoate. In some embodiments, the preceding claims, wherein the pharmaceutical composition further provides a mean plasma CL/F of about 776.4+/−256.9 mL/h of ethyl eicosapentaenoate. In some embodiments, the pharmaceutical composition further provides a mean plasma V_{z}/F of about 79.8+/−62.6 L of ethyl eicosapentaenoate. In some embodiments, the pharmaceutical composition provides a mean RBC Cₘₐₓ of about 42.3+/−14.0 μg/mL, a mean RBC AUC₀₋₂₄ₕ of about 801.5+/−268.5 μg·h/mL, a median RBC Tₘₐₓ of about 12 hours, and a mean RBC T_{1/2} of about 683.8 hours of ethyl eicosapentaenoate. In some embodiments, the pharmaceutical composition further provides a mean RBC Cₘᵢₙ of about 31.2+/−12.2 μg/mL of ethyl eicosapentaenoate. In some embodiments, the pharmaceutical composition further provides a mean plasma Cₘₐₓ of about 0.66+/−0.34 μg/mL, a mean plasma AUC₀₋₂₄ₕ of about 6.9+/−3.1 μg·h/mL, a median plasma Tₘₐₓ of about 5 hours, and a mean plasma T_{1/2} of about 80.8+/−37.5 hours of unesterified eicosapentaenoic acid when administered orally twice per day to a human subject. In some embodiments, the pharmaceutical composition further provides a mean plasma Cₘᵢₙ of about 0.41+/−0.28 μg/mL of unesterified eicosapentaenoic acid. In some embodiments, the pharmaceutical composition further provides a mean plasma CL/F of about 364,513+/−214,003 mL/h of unesterified eicosapentaenoic acid. In some embodiments, the pharmaceutical composition further provides a mean plasma V_{z}/F of about 33,164+/−8,068 L of unesterified eicosapentaenoic acid.

In some embodiments, a pharmaceutical composition comprising about 2 g of ethyl eicosapentaenoate (e.g., about 1600 mg to about 2400 mg, about 1650 mg to about 2350 mg, about 1700 mg to about 2300 mg, about 1750 mg to about 2250 mg, about 1800 mg to about 2200 mg, about 1850 mg to about 2150 mg, about 1900 mg to about 2100 mg, or about 1950 mg to about 2050 mg) provides a mean plasma Cₘₐₓ of about 347.2+/−112.5 μg/mL, a mean plasma AUC₀₋₂₄ₕ of about 6519+/−1963 μg·h/mL, a median plasma Tₘₐₓ of about 5 hours, and a mean plasma T_{1/2} of about 89.3+/−42.0 hours of ethyl eicosapentaenoate when administered orally twice per day to a human subject. In some embodiments, the pharmaceutical composition further provides a mean plasma Cₘᵢₙ of about 101.0+/−50.4 μg/mL of ethyl eicosapentaenoate. In some embodiments, the pharmaceutical composition further provides a mean plasma CL/F of about 683.7+/−280.6 mL/h of ethyl eicosapentaenoate. In some embodiments, the pharmaceutical composition further provides a mean plasma V_{z}/F of about 88.4+/−55.2 L of ethyl eicosapentaenoate. In some embodiments, the pharmaceutical composition further provides a mean RBC Cₘₐₓ of about 76.7+/−25.2 μg/mL, a mean RBC AUC₀₋₂₄ₕ of about 1472+/−469.5 μg·h/mL, a median RBC Tₘₐₓ of about 8 hours, and a mean RBC T_{1/2} of about 371.4+/−311.5 hours of ethyl eicosapentaenoate. In some embodiments, the pharmaceutical composition further provides a mean RBC Cₘᵢₙ of about 64.1+/−21.8 μg/mL of ethyl eicosapentaenoate.

In some embodiments, the pharmaceutical composition provides a mean plasma Cₘₐₓ of about 1.4+/−0.41 μg/mL, a mean plasma AUC₀₋₂₄ₕ of about 18.4+/−4.6 μg·h/mL, a median plasma Tₘₐₓ of about 5 hours, and a mean plasma T_{1/2} of about 97.2+/−36.5 hours of unesterified eicosapentaenoic acid. In some embodiments, the pharmaceutical composition further provides a mean plasma Cₘᵢₙ of about 1.06+/−0.56 μg/mL of unesterified eicosapentaenoic acid. In some embodiments, the pharmaceutical composition further provides a mean plasma CL/F of about 234,329+/−81,639 mL/h of unesterified eicosapentaenoic acid. In some embodiments, the pharmaceutical composition further provides a mean plasma V_{z}/F of about 28,200+/−10,929 L of unesterified eicosapentaenoic acid.

In some embodiments, the pharmaceutical composition is administered in unit dose form, wherein each unit dose includes about 500 mg or about 1000 mg of ethyl eicosapentaenoate. In some embodiments, unit dose forms are administered about 1 to about 4 times per day, for example about 1 time per day, about 2 times per day, about 3 times per day, or about 4 times per day.

### EXAMPLES

### Example 1

### Pharmacokinetics of Pharmaceutical Compositions Comprising Ethyl Eicosapentaenoate

A study to characterize the pharmacokinetics of ethyl eicosapentaenoate in plasma and red blood cells after multiple-dose administration of IPE at 2 g per day and 4 g per day was performed in healthy adult male and female humans.

The study was a phase 1, open-label, randomized, multidose study in healthy, nonsmoking men and women aged >18 and ≦55 years with a body mass index (BMI)>18 and ≦30 kg/m². Exclusion criteria included use within 6 weeks prior to randomization through study end of any lipid-altering medications or supplements, including statins, niacin at levels of greater than 200 mg/day, fibrates, ezetimibe, bile acid sequestrants, omega-3 fatty acid medications, and supplements or foods enriched with omega-3 fatty acids (no more than 2 servings of fish per week were permitted).

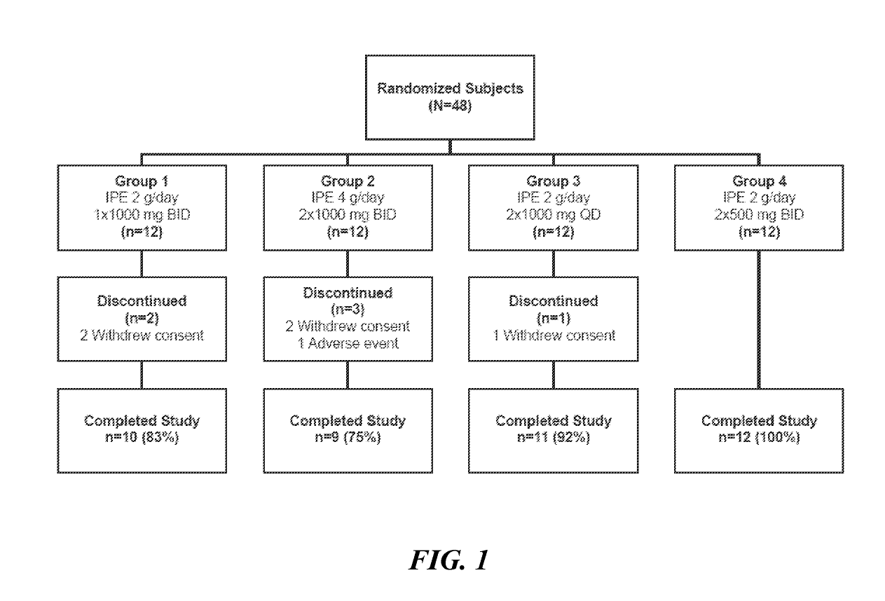

Following a 14-day screening period, subjects entered a 28-day treatment period and were randomized to one of four IPE dose regimen treatment groups as shown in FIG. 1. Group 1 received IPE 2 g daily (1000-mg capsules; twice-daily [BID] regimen); Group 2 received IPE 4 g daily (1000-mg capsules; BID regimen); Group 3 received IPE 2 g daily (1000-mg capsules; once-daily [QD] regimen); and Group 4 received IPE 2 g daily (500-mg capsules; BID regimen). Treatment was administered orally, with or following a meal in the morning and evening for BID regimens, or in the morning only for the QD regimen. Subjects then entered an 18-day post-treatment pharmacokinetic sampling period.

The safety population was defined as all randomized subjects who received at least one dose of study drug. The per-protocol population was defined as all safety population subjects who completed the 28-day treatment period without any major protocol deviations, and who provided blood samples for EPA analyses.

Assessments and Measurements

Fasting EPA concentrations were measured with a validated liquid chromatography with tandem mass spectrometry (LC-MS/MS) method in plasma and RBCs prior to the morning dose (days 1, 14, 26, 28) and at serial time points (1, 3, 5, 6, 8, 10, 12, 24, 48, 72, 120, 192, 312, and 432 hours) after the morning dose on day 28. After the last dose on day 28, EPA concentrations were followed for 18 days to estimate the expected long half-life of EPA. Total plasma EPA included all EPA forms (unesterified EPA and that incorporated in phospholipids, triacylglycerols, and cholesteryl esters). In RBCs, EPA was measured in the cell membrane, where EPA is mainly incorporated in phospholipids.

For total EPA in plasma and RBCs, lipids were isolated by acid/methanol/chloroform extraction followed by centrifugation and purified by isohexane and solid-phase extraction after confirmed complete lipid hydrolysis and transmethylation (acid/methanol, 50° C. overnight). For unesterified EPA in plasma, 25 μL of an inhibitor solution (0.5 g sodium fluoride, 1.0 g L-ascorbic acid, and 0.25 g 5-methylisoxazole-3-carboxylic acid per 10 mL water) was included per 1 mL plasma sample to prevent degradation and lipids were isolated by centrifugation after methanol/chloroform extraction (without hydrolysis or methylation) and further purified by protein precipitation and solid-phase extraction. Quantitation utilized linolenic acid as an internal standard and EPA as the standard for the calibration curve; for total plasma EPA, RBC EPA, and unesterified EPA, the lower limits of quantitation were 10 μg/mL, 5 μg/mL, and 50 ng/mL, respectively. EPA concentrations were measured at Charles River Laboratories Ltd (Elphinstone Research Center, Tranent, Edinburgh, Scotland, UK).

Pharmacokinetic parameters were calculated with standard methods and included area under the plasma concentration versus time curve from time zero to 24 hours (AUC₀₋₂₄ₕᵣ), calculated using the linear trapezoidal rule; maximum observed concentration (Cₘₐₓ); minimum observed plasma concentration (Cₘᵢₙ, pre-dose, trough concentration); apparent terminal elimination half-life (T_{1/2}) calculated from (logₑ2)/λ_{z}, where λ_{z} is the apparent terminal rate constant obtained from the slope of the line, fitted by linear least-squares regression, through the terminal points of the natural log of the concentration versus time plots of these points; apparent total plasma clearance (CL/F) after an oral dose, calculated from Dose/AUC_{0-T}, where F is the oral bioavailability; time of observed Cₘₐₓ (Tₘₐₓ); and apparent volume of distribution after an oral dose (V_{z}/F), calculated from Dose/AUC_{0-T}×λ_{z}, where F is the oral bioavailability. Unless otherwise specified, all reported parameters are based on baseline-subtracted concentrations (baseline is due to EPA derived from dietary sources).

The average daily dose of IPE was calculated based on the unused capsule counts across all treatment days prior to the last day of dosing. Percent compliance was calculated as actual daily dose/planned daily dose×100.

Safety assessments consisted of adverse event (AE) monitoring, clinical laboratory measurements (chemistry, hematology, and urinalysis) 12-lead electrocardiographic measurements, vital signs (systolic and diastolic blood pressure, heart rate, respiratory rate, and oral body temperature) and physical examination findings.

Results—Subjects

A total of 48 subjects were randomized to the 4 treatment groups, with 6 male and 6 female subjects in each group. There were 6 subjects who discontinued the study due to withdrawal of consent (n=5) and an AE (n=1; cholecystitis due to previously undiagnosed, longstanding cholelithiasis unrelated to study drug); 42 subjects completed the study (87.5%) (FIG. 1). Mean age (SD) was 38.8 (11.9) years and subjects were primarily white (n=37; 77.1%). Demographic and other baseline characteristics for subjects in the safety population are presented in Table 1:

TABLE 1
Subject Demographics (Safety Population).

| | Group 1 | Group 2 | Group 3 | Group 4 | Total |
|---|---|---|---|---|---|
| Characteristic | (n = 12) | (n = 12) | (n = 12) | (n = 12) | (n = 48) |
| Age, mean (SD), y | 36.9 (13.5) | 37.9 (12.9) | 39.8 (10.7) | 40.7 (11.5) | 38.8 (11.9) |
| Male, n (%) | 6 (50.0) | 6 (50.0) | 6 (50.0) | 6 (50.0) | 24 (50.0) |
| Race, n (%) | | | | | |
| White | 8 (66.7) | 10 (83.3) | 9 (75.0) | 10 (83.3) | 37 (77.1) |
| Black | 4 (33.3) | 2 (16.7) | 3 (25.0) | 2 (16.7) | 11 (22.9) |
| Ethnicity | | | | | |
| Hispanic | 9 (75.0) | 9 (75.0) | 8 (66.7) | 8 (66.7) | 34 (70.8) |
| Weight, mean (SD), kg | 74.5 (14.1) | 75.5 (13.4) | 72.9 (11.0) | 73.1 (12.1) | 74.0 (12.4) |
| BMI, mean (SD), kg/m² | 26.2 (2.6) | 27.2 (2.6) | 26.1 (2.5) | 25.7 (3.0) | 26.3 (2.6) |

Baseline total EPA plasma concentrations were variable between subjects, with a study-wide mean (SD) of 15.3 (15.2) μg/mL. Fourteen subjects had baseline total EPA values lower than the lower limit of quantitation (10 μg/mL). The study-wide mean (SD) baseline unesterified EPA plasma concentration was 0.099 (0.095) μg/mL, which is less than 1% of the total EPA plasma concentration. In RBCs, the study-wide mean (SD) baseline unesterified EPA concentration was 8.01±9.89 μg/mL.

Mean daily IPE dose was 2 g for all groups except Group 2, which had a mean daily IPE dose of 3.9 g. Compliance rates were 99.4%, 97.1%, 99.1%, and 101.3% for Group 1 through 4, respectively.

Pharmacokinetic Results

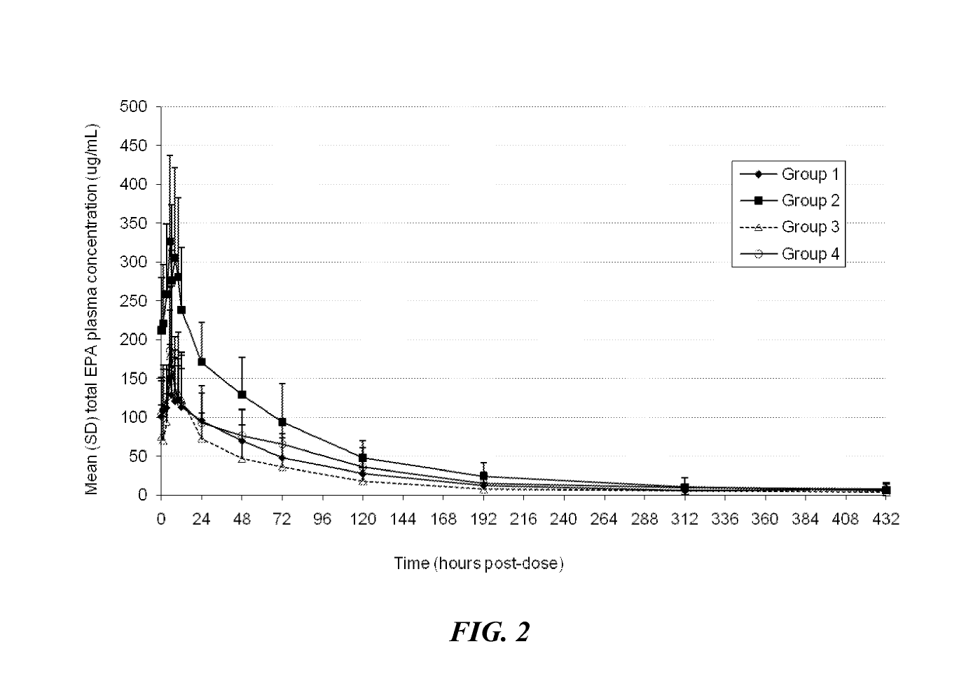

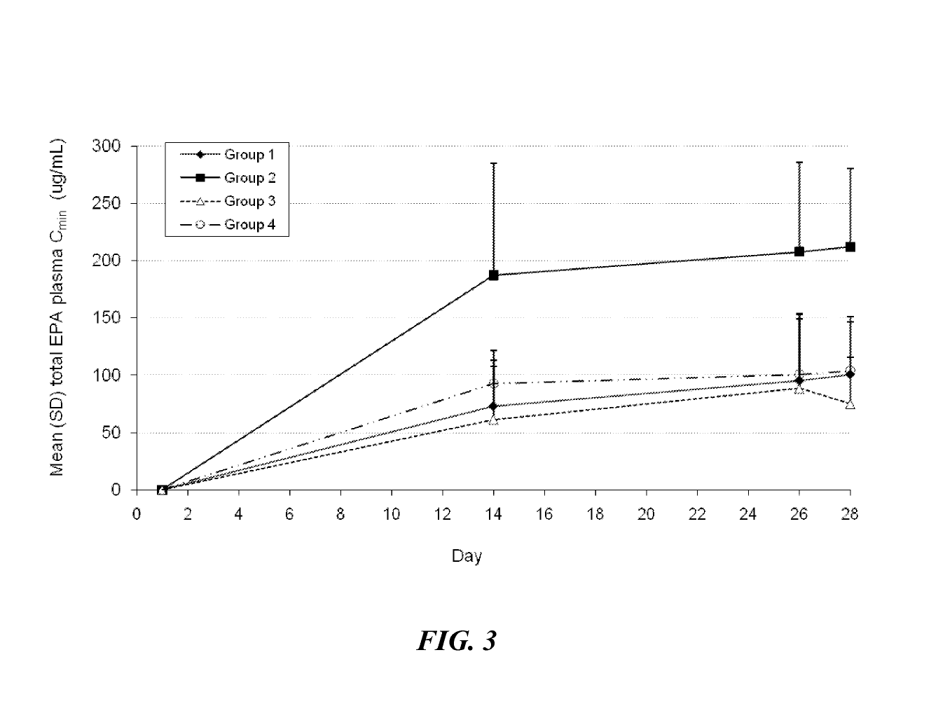

Following IPE dosing, maximum concentration of total EPA was reached in approximately 5 to 6 hours in plasma (Table 2, FIG. 2) and approximately 8 to 24 hours in RBCs (Table 2). The mean terminal half-life of total EPA in plasma was long, ranging between 70 and 89 hours for the 4 treatment groups. The mean oral clearance and volume of distribution of total EPA in plasma ranged between 684 and 868 mL/hr and 79 and 88 L, respectively (Table 2). Steady state was reached by day 14 in plasma in all treatment groups (FIG. 3). However, RBC concentrations slowly increased over the duration of treatment, and steady state was not reached by day 28 (data not shown). Study-wide steady-state means (SD) for half-life (mainly from plasma lipids), total plasma clearance, and volume of distribution of total EPA were 79 (47) hours, 757 (283) mL/hr, and 82 (56) L, respectively.

TABLE 2
Pharmacokinetic parameters by treatment at day 28 (per-protocol population).

| | | | | Group 2: | | | | | |
|---|---|---|---|---|---|---|---|---|---|
| | | Group 1: | | IPE 4 g/d | | Group 3: | | Group 4: | |
| | | IPE 2 g/d | | 2 × 1000 mg | | IPE 2 g/d | | IPE 2 g/d | |
| | PK | 1 × 1000 mg BID | | BID | | 2 × 1000 mg QD | | 2 × 500 mg BID | |
| Analyte | parameter* | (n = 10) | | (n = 9) | | (n = 12) | | (n = 12) | |
| Total EPA in | Baseline | 7.9 | (7.0) | 19.3 | (16.1) | 19.2 | (17.5) | 14.7 | (16.6) |
| plasma | (μg/mL) | | | | | | | | |
| | Cₘₐₓ (μg/mL) | 154.9 | (49.4) | 347.2 | (112.5) | 232.8 | (127.6) | 210.5 | (93.1) |
| | AUC_{0-24 hr} | 2907 | (1160) | 6519 | (1963) | 2659 | (1136) | 3233 | (1104) |
| | (μg · hr/mL) | | | | | | | | |
| | Cₘᵢₙ (μg/mL) | 101.0 | (50.4) | 211.9 | (68.2) | 75.1 | (40.5) | 104.2 | (42.4) |
| | Tₘₐₓ (hr) | 5 | (5, 8) | 5 | (3, 8) | 5 | (3, 12) | 6 | (5, 8) |
| | T_{1/2} (hr) | 75.1 | (46.5) | 89.3 | (42.0) | 69.7 | (60.9) | 83.5 | (38.8) |
| | CL/F (mL/hr) | 776.4 | (256.9) | 683.7 | (280.6) | 867.7 | (328.8) | 684.5 | (248.3) |
| | V_{z}/F (L) | 79.8 | (62.6) | 88.4 | (55.2) | 79.3 | (71.5) | 79.3 | (35.3) |
| Total EPA in | Baseline | 5.7 | (4.3) | 12.1 | (15.7) | 8.2 | (7.1) | 6.7 | (10.4) |
| RBC | (μg/mL) | | | | | | | | |
| | Cₘₐₓ (μg/mL) | 42.3 | (14.0) | 76.7 | (25.2) | 31.0 | (11.1) | 37.6 | (15.7) |
| | AUC_{0-24 hr} | 801.5 | (268.5) | 1472 | (469.5) | 557.6 | (239.7) | 573.9 | (336.4) |
| | (μg · hr/mL) | | | | | | | | |
| | Cₘᵢₙ (μg/mL) | 31.2 | (12.2) | 64.1 | (21.8) | 20.7 | (8.6) | 26.3 | (17.7) |
| | Tₘₐₓ (hr) | 12 | (1, 72) | 8 | (1, 12) | 10 | (6, 48) | 24 | (0, 120) |
| | T_{1/2} (hr) | 683.8 | (NC) | 371.4 | (311.5) | 303.2 | (NC) | 314.4 | (194.5) |
| Unesterified | Baseline | 0.13 | (0.12) | 0.08 | (0.05) | 0.09 | (0.06) | 0.10 | (0.13) |
| EPA in plasma | (μg/mL) | | | | | | | | |
| | Cₘₐₓ (μg/mL) | 0.66 | (0.34) | 1.4 | (0.41) | 1.4 | (0.49) | 0.70 | (0.24) |
| | AUC_{0-24 hr} | 6.9 | (3.1) | 18.4 | (4.6) | 9.0 | (2.5) | 7.5 | (2.6) |
| | (μg · hr/mL) | | | | | | | | |
| | Cₘᵢₙ (μg/mL) | 0.41 | (0.28) | 1.06 | (0.56) | 0.36 | (0.22) | 0.44 | (0.24) |
| | Tₘₐₓ (hr) | 5 | (0, 48) | 5 | (0, 24) | 5 | (3, 8) | 3 | (0, 24) |
| | T_{1/2} (hr) | 80.8 | (37.5) | 97.2 | (36.5) | 136.3 | (80.8) | NC | |
| | CL/F (mL/hr) | 364,513 | (214,003) | 234,329 | (81,639) | 241,855 | (82,203) | 326,622 | (207,574) |

| | | | | | | | |
|---|---|---|---|---|---|---|---|
| V_{z}/F (L) | 33,164 | (8068) | 28,200 | (10,929) | 40,873 | (19,580) | NC |

A small fraction (≦0.5%) of the total plasma EPA was determined to be unesterified. Based on AUC₀₋₂₄ₕᵣ in plasma, 0.3% of the total EPA was unesterified in Group 1 (2 g/day) and Group 2 (4 g/day); based on maximum concentration in plasma, 0.5% and 0.4% of the total EPA was unesterified EPA in group 1 (2 g/day) and group 2 (4 g/day), respectively.

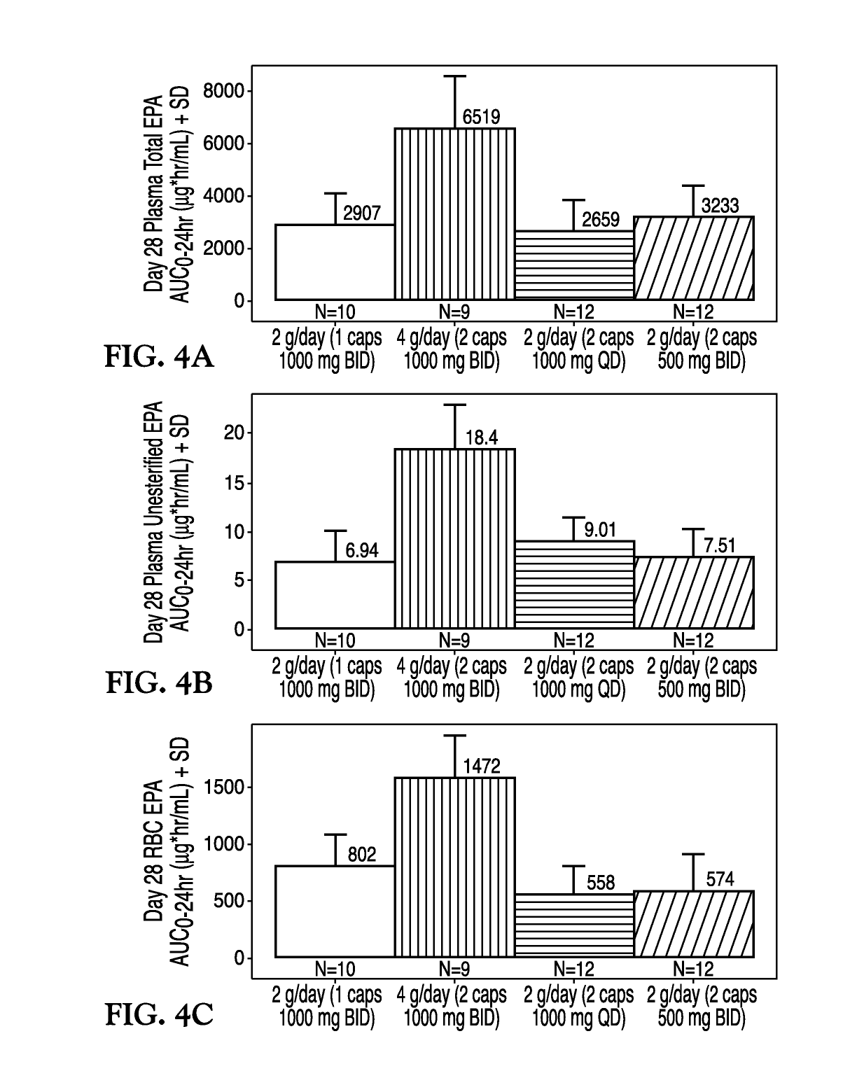

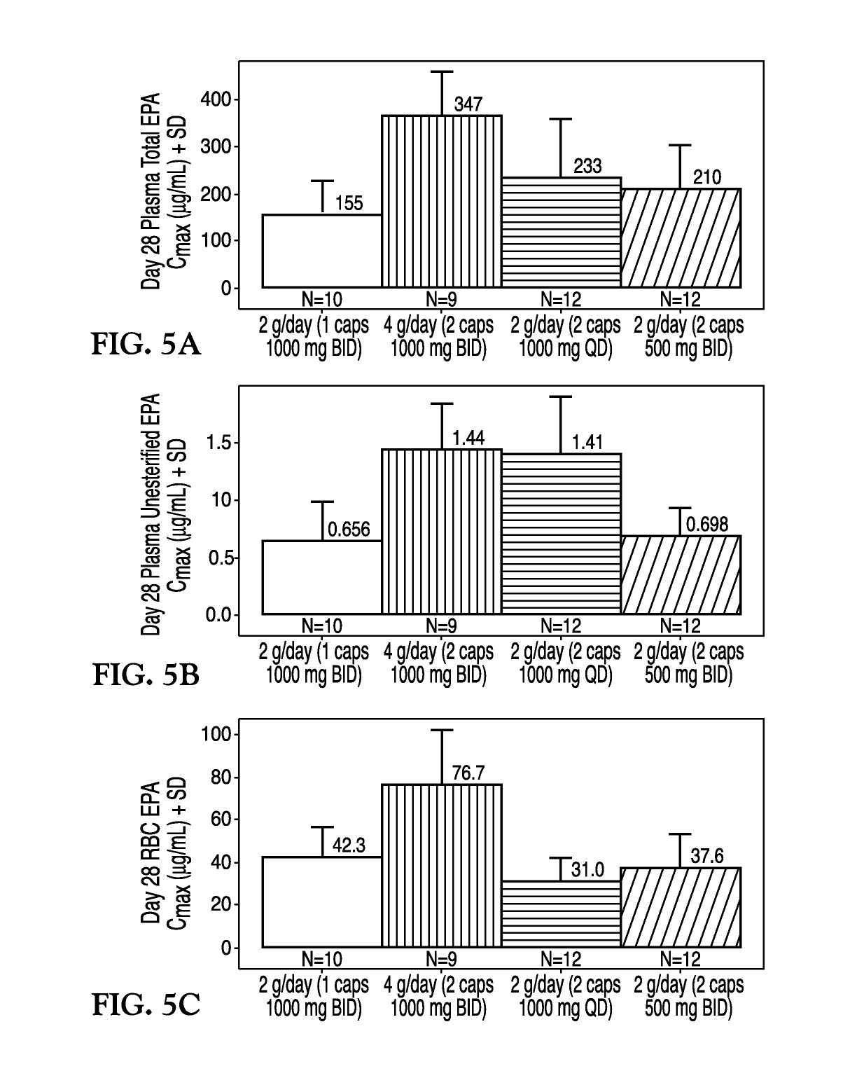

Comparisons of exposure (AUC₀₋₂₄ₕᵣ and Cₘₐₓ) revealed similarity between QD and BID regimens and between 1×1000-mg and 2×500-mg formulations (FIGS. 4 and 5; Group 1 vs. Group 3, AUC₀₋₂₄ₕᵣ for total plasma, total RBCs, and unesterified EPA, with observed differences in maximum concentration expected based on dosing regimen; Group 1 vs. Group 4, AUC₀₋₂₄ₕᵣ and maximum concentration for total plasma and unesterified EPA). Dose linearity was observed between IPE 2 g/day and 4 g/day (Group 1 vs. Group 2; dose-normalized comparison of AUC and maximum concentration for total plasma, total RBCs, and unesterified EPA). Based on graphical examination of the AUC₀₋₂₄ₕᵣ and maximum concentration of total and unesterified plasma EPA concentrations, no effect of age was observed. Minimum concentration appeared to be slightly lower for males compared with females in some treatment groups, but overall, no consistent gender effect was observed based on all remaining exposure parameters.

Safety and Tolerability

Fourteen (29.2%) subjects reported at least one adverse event (“AE”) during the study (3 [25.0%] in Group 1, 4 [33.3%] in Group 2, 3 [25.0%] in Group 3, and 4 [33.3%] in Group 4). All AEs were treatment emergent and mild or moderate in intensity. The most common AEs were upper respiratory tract infection (two subjects in Group 2) and headache (two subjects in Group 3). One subject in Group 2 discontinued study treatment as noted earlier due to cholecystitis, which was considered unrelated to study drug. There were no clinically meaningful changes in laboratory, electrocardiographic, or physical examination findings.

Discussion

The present study describes the plasma and RBC pharmacokinetics of IPE, a high-purity prescription form of EPA ethyl ester. Once-daily versus twice-daily regimens, 500-mg versus 1000-mg formulations, and 2 g/day versus 4 g/day doses were compared, and the effects of age and gender were also evaluated.

Following oral dosing with IPE 4 g/day and 2 g/day, the study-wide mean (SD) elimination half-life of total EPA at steady state was 79 (47) hours. The study-wide mean apparent total plasma clearance of total EPA at steady state was 757 (283) mL/hr and the apparent volume of distribution of total EPA at steady state was 82 (56) L. Maximum concentrations of total EPA were attained approximately 5 to 6 hours in plasma after dosing. Maximum EPA concentrations occurred later in RBCs compared with plasma (˜8-24 hours after dosing). Steady-state concentrations of total plasma EPA were observed 14 days after continuous dosing, whereas steady state was not reached by day 28 in RBCs.

The slow increase in RBC concentrations observed over the duration of treatment in the present study was consistent with the slow washout of EPA after cessation of dosing (long elimination half-life) in RBCs and likely due to the known slower process of incorporation of EPA into RBC membranes. The maximum plasma concentration of 5 to 6 hours post dosing observed in this study are in agreement with that of a prescription formulation of ethyl EPA available in Japan. The study-wide mean elimination half-life of total plasma EPA of 79 hours was consistent with previously reported mean elimination half-lives ranging from approximately 1 to 3 days for plasma EPA in phospholipids, cholesteryl esters, and triacylglycerol.

Based on dose-normalized maximum observed concentration and AUC of total and unesterified EPA in plasma and total EPA in RBCs, the mean exposure to EPA appears to be dose-proportional between IPE 2 and 4 g/day. This pharmacokinetic dose-linearity was supported by the statistical comparisons of dose-normalized pharmacokinetic values, indicating that IPE has predictable pharmacokinetics. Administration of prescription omega-3-acid ethyl esters (EPA plus DHA) has also been shown to result in dose-dependent increases in serum EPA, but increases in serum DHA were found to be less pronounced and not dose-dependent.

In general, the once-daily and twice-daily dosing regimens for the same daily dose of IPE resulted in similar total AUC for total EPA in plasma. As expected, the maximum observed concentration was higher and the trough concentration was lower in the once-daily regimen compared with the twice-daily regimen. As is typical for drugs with long half-lives, the major determinant of steady-state exposure to EPA in plasma, and even more so in RBCs, was the total daily dose of IPE, irrespective of how the dose was divided over the course of the day. The relatively long half-life of EPA in plasma permits a dosing schedule with intervals of hours, and day-to-day variability in the dosing interval is not expected to influence the average plasma or tissue exposure to EPA. In comparing the 500-mg and 1000-mg formulations of IPE at the same daily dose, similar total plasma EPA maximum observed concentrations and AUCs were observed, indicating that, as expected, the 500-mg and 1000-mg formulations provide comparable exposures at comparable doses.

### Example 2

### ANCHOR Study

A multi-center, placebo-controlled, randomized, double-blind, 12-week study was performed to evaluate the efficacy and safety of >96% E-EPA in patients with fasting triglyceride levels ≧200 mg/dl and <500 mg/dl despite statin therapy (the mean of two qualifying entry values needed to be ≧185 mg/dl and at least one of the values needs to be ≧200 mg/dl). The primary objective of the study was to determine the efficacy of >96% E-EPA 2 g daily and 4 g daily, compared to placebo, in lowering fasting TG levels in patients with high risk for cardiovascular disease and with fasting TG levels ≧200 mg/dL and <500 mg/dL, despite treatment to LDL-C goal on statin therapy.

The secondary objectives of this study were the following:
To determine the safety and tolerability of >96% E-EPA 2 g daily and 4 g daily;
To determine the effect of >96% E-EPA on lipid and apolipoprotein profiles including total cholesterol (TC), non-high-density lipoprotein cholesterol (non-HDL-C), low density lipoprotein cholesterol (LDL-C), high density lipoprotein cholesterol (HDL-C), and very high density lipoprotein cholesterol (VHDL-C);
To determine the effect of >96% E-EPA (on lipoprotein associated phospholipase A₂ (Lp-PLA₂) from baseline to week 12;
To determine the effect of >96% E-EPA on low-density lipoprotein (LDL) particle number and size;
To determine the effect of >96% E-EPA on oxidized LDL;
To determine the effect of >96% E-EPA on fasting plasma glucose (FPG) and hemoglobin A_{1c} (HbA_{1c});
To determine the effect of >96% E-EPA on insulin resistance;
To determine the effect of >96% E-EPA on high-sensitivity C-reactive protein (hsCRP);
To determine the effects of >96% E-EPA 2 g daily and 4 g daily on the incorporation of fatty acids into red blood cell membranes and into plasma phospholipids;
To explore the relationship between baseline fasting TG levels and the reduction in fasting TG levels; and
To explore the relationship between changes of fatty acid concentrations in plasma and red blood cell membranes, and the reduction in fasting TG levels.

The population for this study was men and women >18 years of age with a body mass index ≦45 kg/m² with fasting TG levels greater than or equal to 200 mg/dl and less than 500 mg/dl and on a stable does of statin therapy (with or without ezetimibe). The statin must have been atorvostatin, rosuvastatin or simvastatin. The dose of statin must have been stable for ≧4 weeks prior to the LDL-C/TG baseline qualifying measurement for randomization. The statin dose was optimal such that the patients are at their LDL-C goal at the LDL-C/TG baseline qualifying measurements. The same statin at the same dose was continued until the study ended.

Patients taking any additional non-statin, lipid-altering medications (niacin >200 mg/day, fibrates, fish oil, other products containing omega-3 fatty acids, or other herbal products or dietary supplements with potential lipid-altering effects), either alone or in combination with statin therapy (with or without ezetimibe), must have been able to safely discontinue non-statin, lipid-altering therapy at screening.

Patients at high risk for CVD, i.e., patients with clinical coronary heart disease (CHD) or clinical CHD risk equivalents (10-year risk >20%) as defined in the National Cholesterol Education Program (NCEP) Adult Treatment Panel III (ATP III) Guidelines were eligible to participate in this study. Those included patients with any of the following criteria: (1) Known CVD, either clinical coronary heart disease (CHD), symptomatic carotid artery disease (CAD), peripheral artery disease (PAD) or abdominal aortic aneurism; or (2) Diabetes Mellitus (Type 1 or 2).

Approximately 648 patients were randomized at approximately 80 centers in the U.S. The study was a 18- to 20-week, Phase 3, multi-center study consisting of 2 study periods: (1) A 6- to 8-week screening period that included a diet and lifestyle stabilization, a non-statin lipid-altering treatment washout, and an LDL-C and TG qualifying period and (2) A 12-week, double-blind, randomized, placebo-controlled treatment period.

During the screening period and double-blind treatment period, all visits were to be within ±3 days of the scheduled time. All patients continued to take the statin product (with or without ezetimibe) at the same dose they were taking at screening throughout their participation in the study.

The 6- to 8-week screening period included a diet and lifestyle stabilization, a non-statin lipid-altering treatment washout, and an LDL-C and TG qualifying period. The screening visit (Visit 1) occurred for all patients at either 6 weeks (for patients on stable statin therapy—with or without ezetimibe—at screening) or 8 weeks (for patients who required washout of their then-current non-statin lipid-altering therapy at screening) before randomization, as follows:
Patients who did not require a washout: The screening visit occurred at Visit 1 (Week −6). Eligible patients entered a 4-week diet and lifestyle stabilization period. At the screening visit, all patients received counseling regarding the importance of the National Cholesterol Education Program (NCEP) Therapeutic Lifestyle Changes (TLC) diet and received basic instructions on how to follow this diet.
Patients who required a washout: The screening visit occurred at Visit 1 (Week −8). Eligible patients began a 6-week washout period at the screening visit (i.e. 6 weeks washout before the first LDL-C/TG qualifying visit). Patients received counseling regarding the NCEP TLC diet and received basic instructions on how to follow this diet. Site personnel contacted patients who did not qualify for participation based on screening laboratory test results to instruct them to resume their prior lipid-altering medications.

At the end of the 4-week diet and lifestyle stabilization period or the 6-week diet and stabilization and washout period, eligible patients entered the 2-week LDL-C and TG qualifying period and had their fasting LDL-C and TG levels measured at Visit 2 (Week −2) and Visit 3 (Week −1). Eligible patients must have had an average fasting LDL-C level ≧40 mg/dL and <100 mg/dL and an average fasting TG level ≧200 mg/dL and <500 mg/dL to enter the 12-week double-blind treatment period. The LDL-C and TG levels for qualification were based on the average (arithmetic mean) of the Visit 2 (Week −2) and Visit 3 (Week −1) values. If a patient's average LDL-C and/or TG levels from Visit 2 and Visit 3 fall outside the required range for entry into the study, an additional fasting lipid profile was collected 1 week later at Visit 3.1. If a third sample was collected at Visit 3.1, entry into the study was based on the average (arithmetic mean) of the values from Visit 3 and Visit 3.1.

After confirmation of qualifying fasting LDL-C and TG values, eligible patients entered a 12-week, randomized, double-blind treatment period. At Visit 4 (Week 0), patients were randomly assigned to 1 of the following treatment groups:
>96% E-EPA 2 g daily,
>96% E-EPA 4 g daily, or
Placebo.

Approximately 216 patients per treatment group were randomized in this study. Stratification was by type of statin (atorvastatin, rosuvastatin or simvastatin), the presence of diabetes, and gender. Baseline parameters for randomized subjects in this study are shown in Table 3 below. The lower-efficacy statin regimen included administration of 5-10 mg of simvastatin per day; the medium-efficacy statin regimens included administration of 5-10 mg rosuvastatin per day, 10-20 mg of atorvastatin per day, 20-40 mg of simvastatin per day, or 10-20 mg of simvastatin and 5-10 mg of ezetimibe per day; and the higher-efficacy statin regimens included administration of 20-40 mg of rosuvastatin per day, 40-80 mg of atorvastatin per day, 80 mg of simvastatin per day, or 40-80 mg of simvastatin and 5-10 mg of ezetimibe per day.

TABLE 3
Patient Characteristics (Randomized Population)

| | AMR101 | AMR101 | |
|---|---|---|---|
| | 4 g/day | 2 g/day | Placebo |
| | (n = 233) | (n = 236) | (n = 233) |
| Age, y, mean (SD) | 61.1 (10.03) | 61.8 (9.42) | 61.2 (10.05) |
| Male, n (%) | 142 (60.9) | 144 (61.0) | 145 (62.2) |
| White, n (%) | 226 (97.0) | 226 (95.8) | 224 (96.1) |
| Weight, kg, mean (SD) | 94.5 (18.30) | 95.5 (18.29) | 97.0 (19.14) |
| BMI, kg/m², mean (SD) | 32.7 (4.99) | 32.9 (4.98) | 33.0 (5.04) |
| Diabetes, n (%) | 171 (73.4) | 172 (72.9) | 171 (73.4) |
| Baseline TG >750 mg/dL, | NA | NA | NA |
| n (%) | | | |

Statin use, n (%):

| | | | |
|---|---|---|---|
| Any | 233 (100) | 236 (100) | 233 (100) |
| Atorvastatin | 44 (18.9) | 43 (18.2) | 45 (19.3) |
| Simvastatin | 134 (57.5) | 136 (57.6) | 133 (57.1) |
| Rosuvastatin | 55 (23.6) | 57 (24.2) | 55 (23.6) |

Statin efficacy regimens, n (%)

| | | | |
|---|---|---|---|
| Lower | 16 (6.9) | 17 (7.2) | 15 (6.4) |
| Medium | 148 (63.5) | 148 (62.7) | 144 (61.8) |
| Higher | 69 (29.6) | 71 (30.1) | 74 (31.8) |

Baseline lipid parameters for subjects in the intent-to-treat (“ITT”) population of this study are shown in Table 4 below. Data are presented as median (interquartile range) values.

TABLE 4
Baseline Lipid Parameters (ITT Population)

| | AMR101 | AMR101 | |
|---|---|---|---|
| | 4 g/day | 2 g/day | Placebo |
| Lipid Parameter | (IQR) | (IQR) | (IQR) |
| (mg/dL) | (n = 233) | (n = 236) | (n = 233) |
| TG | 264.8 (93.0) | 254.0 (92.5) | 259.0 (81.0) |
| | (n = 226) | (n = 234) | (n = 227) |
| LDL-C | 82.0 (25.0) | 82.0 (24.0) | 84.0 (27.0) |
| | (n = 225) | (n = 233) | (n = 226) |
| Non-HDL-C | 128.0 (32.0) | 128.0 (33.0) | 128.0 (34.0) |
| | (n = 226) | (n = 234) | (n = 227) |
| TC | 167.0 (38.0) | 169.0 (34.0) | 168.0 (38.0) |
| | (n = 226) | (n = 234) | (n = 227) |
| HDL-C | 37.0 (12.0) | 38.0 (13.0) | 39.0 (12.0) |
| | (n = 226) | (n = 234) | (n = 227) |

During the double-blind treatment period, patients returned to the site at Visit 5 (Week 4), Visit 6 (Week 11), and Visit 7 (Week 12) for efficacy and safety evaluations.

Eligible patients were randomly assigned at Visit 4 (Week 0) to receive orally >96% E-EPA 2 g daily, >96% E-EPA 4 g daily, or placebo.

>96% E-EPA was provided in 1 g liquid-filled, oblong, gelatin capsules. The matching placebo capsule was filled with light liquid paraffin and contains 0 g of >96% E-EPA. >96% E-EPA capsules were to be taken with food (i.e. with or at the end of a meal).

During the double-blind treatment period, patients took 2 capsules (>96% E-EPA or matching placebo) in the morning and 2 capsules in the evening for a total of 4 capsules per day.

Patients in the >96% E-EPA 2 g/day treatment group received 1>96% E-EPA 1 g capsule and 1 matching placebo capsule in the morning and in the evening.

Patients in the >96% E-EPA 4 g/day treatment group received 2>96% E-EPA 1 g capsules in the morning and evening.

Patients in the placebo group received 2 matching placebo capsules in the morning and evening.

The primary efficacy variable for the double-blind treatment period was percent change in TG from baseline to Week 12 endpoint. The secondary efficacy variables for the double-blind treatment period included the following:
Percent changes in total cholesterol (TC), high-density lipoprotein cholesterol (HDL-C), LDL-C, calculated non-HDL-C, and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A-I/apo B ratio from baseline to Week 12;
Percent changes in lipoprotein(a) from baseline to Week 12;
Percent changes in LDL particle number and size, measured by nuclear magnetic resonance, from baseline to Week 12;
Percent change in remnant-like particle cholesterol from baseline to Week 12;
Percent change in oxidized LDL from baseline to Week 12;
Changes in FPG and HbA_{1c} from baseline to Week 12;
Change in insulin resistance, as assessed by the homeostasis model index insulin resistance, from baseline to Week 12;
Percent change in lipoprotein associated phospholipase A₂ (Lp-PLA₂) from baseline to Week 12;
Change in intracellular adhesion molecule-1 from baseline to Week 12;
Change in interleukin-2 from baseline to Week 12;
Change in plasminogen activator inhibitor-1 from baseline to Week 12. Note: this parameter will only be collected at sites with proper storage conditions;
Change in hsCRP from baseline to Week 12; and
Change in plasma concentration and red blood cell membrane content of fatty acid from baseline to Week 12 including EPA, docosapentaenoic acid (DPA), docosahexaenoic acid (DHA), arachidonic acid (AA), dihomo-γ-linolenic acid (DGLA), the ratio of EPA/AA, ratio of oleic acid/stearic acid (OA/SA), and the ratio of total omega-3 acids over total omega-6 acids.

Safety assessments included adverse events, clinical laboratory measurements (chemistry, hematology, and urinalysis), 12-lead electrocardiograms (ECGs), vital signs, and physical examinations.

For TG, TC, HDL-C, LDL-C, calculated non-HDL-C, and VLDL-C, baseline was defined as the average of Visit 4 (Week 0) and the preceding lipid qualifying visit (either Visit 3 [Week −1] or if it occurs, Visit 3.1) measurements. Baseline for all other efficacy parameters was the Visit 4 (Week 0) measurement.

For TG, TC, HDL-C, LDL-C, calculated non-HDL-C, and VLDL-C, Week 12 endpoint was defined as the average of Visit 6 (Week 11) and Visit 7 (Week 12) measurements.

Week 12 endpoint for all other efficacy parameters was the Visit 7 (Week 12) measurement.

The primary efficacy analysis was performed using a 2-way analysis of covariance (ANCOVA) model with treatment as a factor and baseline TG value as a covariate. The least-squares mean, standard error, and 2-tailed 95% confidence interval for each treatment group and for each comparison was estimated. The same 2-way ANCOVA model was used for the analysis of secondary efficacy variables.

The primary analysis was repeated for the per-protocol population to confirm the robustness of the results for the intent-to-treat population.

Non-inferiority tests for percent change from baseline in LDL-C were performed between >96% E-EPA doses and placebo using a non-inferiority margin of 6% and a significant level at 0.05.

For the following key secondary efficacy parameters, treatment groups were compared using Dunnett's test to control the Type 1 error rate: TC, LDL-C, HDL-C, non-HDL-C, VLDL-C, Lp-PLA₂, and apo B. For the remaining secondary efficacy parameters, Dunnett's test was not used and the ANCOVA output was considered descriptive.

The evaluation of safety was based primarily on the frequency of adverse events, clinical laboratory assessments, vital signs, and 12-lead ECGs. The primary efficacy variable was the percent change in fasting TG levels from baseline to Week 12. A sample size of 194 completed patients per treatment group provided 90.6% power to detect a difference of 15% between >96% E-EPA and placebo in percent change from baseline in fasting TG levels, assuming a standard deviation of 45% in TG measurements and a significance level of p<0.05.

Previous data on fasting LDL-C showed a difference in percent change from baseline of 2.2%, with a standard deviation of 15%, between study drug and placebo. A sample size of 194 completed patients per treatment group provided 80% power to demonstrate non-inferiority (p<0.05, one-sided) of the LDL-C response between >96% E-EPA 4 g daily and placebo, within a 6% margin. To accommodate a 10% drop-out rate from randomization to completion of the double-blind treatment period, a total of 648 randomized patients were planned (216 patients per treatment group).

A summary of baseline values, end-of-treatment values, and median placebo-adjusted percent change from baseline to study end of inflammation-associated end points in each of the three ITT sub-populations is shown in Table 5.

TABLE 5
Median Placebo-adjusted Percent Change From Baseline to Study End in Inflammation-associated End Points (ITT Populations).

| | | | | | | | | | | Median Placebo- | |
|---|---|---|---|---|---|---|---|---|---|---|---|
| | | | | | | | | | | adjusted % | |
| | AMR101 4 g/day | | | AMR101 2 g/day | | | Placebo | | | Change From Baseline | |
| | Base- | End-of- | Change | Base- | End-of- | Change | Base- | End-of- | Change | AMR101 | AMR101 |
| | line | treatment | from | line | treatment | from | line | treatment | from | 4 g/day | 2 g/day |
| | value | value | baseline, | value | value | baseline, | value | value | baseline, | vs pla- | vs pla- |
| | (IQR) | (IQR) | % (IQR) | (IQR) | (IQR) | % (IQR) | (IQR) | (IQR) | % (IQR) | cebo, P | cebo, P |
| MARINE | n = 76 | | | n = 73 | | | n = 75 | | | | |
| ICAM-1 (ng/mL) | 250.0 | 253.0 | −0.9 | 255.5 | 257.5 | −0.4 | 247.5 | 246.0 | 2.6 | −2.5 | −2.3 |
| (n = 75, 70, 72) | (85.00) | (89.00) | (10.69) | (86.00) | (102.00) | (12.29) | (101.00) | (89.50) | (13.43) | 0.1188 | 0.2201 |
| Ox-LDL (U/L) | 78.8 | 74.8 | −3.0 | 76.2 | 75.2 | 0.4 | 74.3 | 74.8 | 3.3 | −6.4 | −3.0 |
| (n = 74, 70, 71) | (26.51) | (26.52) | (2.35) | (24.00) | (23.52) | (2.41) | (24.86) | (23.32) | (2.39) | 0.0599 | 0.3818 |
| Lp-PLA₂ (ng/mL) | 246.0 | 201.0 | −17.1 | 235.0 | 220.5 | −5.1 | 253.0 | 256.0 | −2.4 | −13.6 | −5.1 |
| (n = 73, 70, 70) | (116.00) | (100.00) | (24.43) | (106.00) | (101.00) | (24.14) | (126.00) | (146.00) | (29.35) | 0.0003 | 0.1529 |
| IL-6 (pg/mL) | 2.3 | 2.4 | 0.3 | 3.0 | 3.0 | 3.4 | 2.5 | 2.3 | 5.3 | 11.0 | 4.7 |
| (n = 60, 62, 61) | (3.34) | (3.32) | (93.07) | (2.78) | (5.39) | (76.70) | (4.12) | (1.85) | (79.27) | 0.3629 | 0.6654 |
| hsCRP (mg/L) | 2.2 | 2.2 | −2.5 | 2.0 | 2.4 | 25.1 | 1.8 | 2.5 | 33.3 | −36.0 | −10.1 |
| (n = 75, 70, 72) | (3.10) | (2.90) | (81.19) | (2.70) | (3.20) | (96.43) | (3.05) | (3.95) | (80.49) | 0.0012 | 0.4028 |
| ANCHOR | n = 226 | | | n = 234 | | | n = 227 | | | | |
| ICAM-1 (ng/mL) | 273.0 | 270.0 | 0.8 | 267.0 | 268.5 | 0.5 | 269.0 | 257.0 | 3.6 | −2.4 | −2.2 |
| (n = 78, 74, 83) | (96.00) | (110.00) | (13.27) | (97.00) | (89.00) | (12.41) | (122.00) | (131.00) | (12.07) | 0.1888 | 0.1944 |
| Ox-LDL (U/L) | 54.0 | 51.4 | −4.8 | 54.0 | 55.8 | 2.6 | 51.8 | 59.7 | 11.6 | −13.3 | −5.8 |
| (n = 78, 75, 84) | (14.60) | (17.50) | (19.63) | (17.80) | (22.80) | (18.28) | (16.80) | (18.10) | (28.09) | 0.0001 | 0.0946 |
| Lp-PLA₂ (ng/mL) | 180.0 | 160.0 | −12.8 | 190.0 | 183.5 | −1.8 | 185.0 | 200.0 | 6.7 | −19.0 | −8.0 |
| (n = 217, 224, | (56.00) | (57.00) | (18.52) | (55.50) | (57.50) | (23.11) | (58.00) | (71.00) | (24.03) | <0.0001 | <0.0001 |
| 213) | | | | | | | | | | | |
| IL-6 (pg/mL) | 2.7 | 2.6 | 3.1 | 2.4 | 2.7 | 6.9 | 3.2 | 2.9 | 3.3 | −1.0 | 7.0 |
| (n = 78, 74, 83) | (2.61) | (2.08) | (56.47) | (2.01) | (2.28) | (51.85) | (3.23) | (2.95) | (59.37) | 0.9031 | 0.3643 |
| hsCRP (mg/L) | 2.2 | 2.0 | −2.4 | 1.9 | 2.5 | 10.3 | 2.2 | 2.6 | 17.1 | −22.0 | −6.8 |
| (n = 217, 227, | (2.70) | (3.00) | (62.75) | (2.90) | (3.40) | (88.61) | (4.00) | (4.70) | (107.99) | 0.0005 | 0.2889 |
| 219) | | | | | | | | | | | |

Only subsets with non-missing baseline and week 12 values are included. Data are presented as median (interquartile range) for end point values.
End Point Abbreviations: hsCRP, high-sensitivity C-reactive protein; ICAM-1, intracellular adhesion molecule-1; IL-6, interleukin-6; IQR, interquartile range; Lp-PLA₂, lipoprotein-associated phospholipase A₂; Ox-LDL, oxidized low-density lipoprotein.

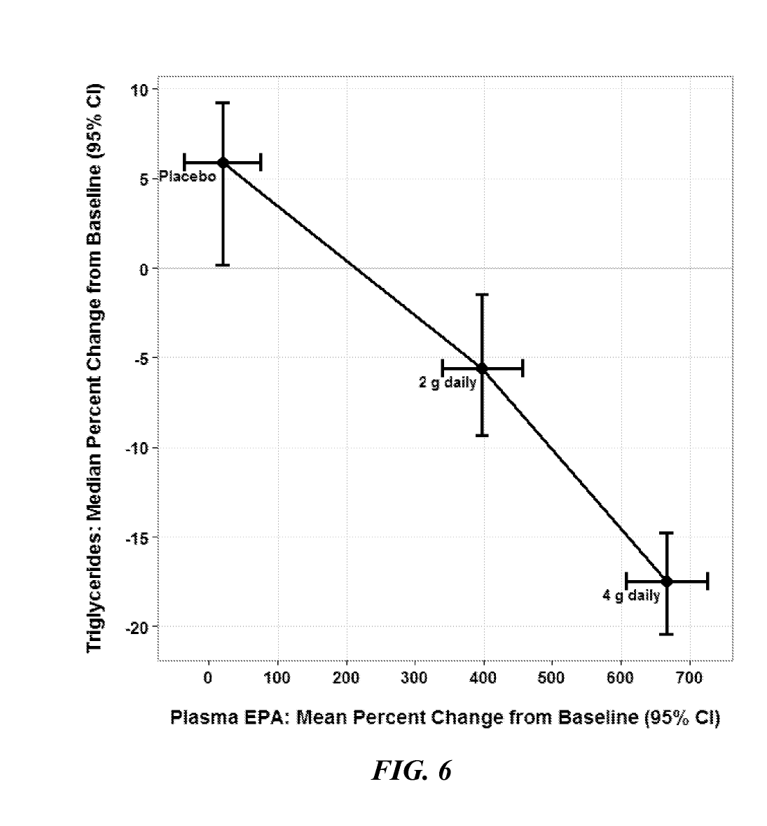

As shown in FIG. 6, a linear concentration-response pharmacodynamics relationship between EPA plasma levels and triglyceride reduction was also observed. Error bars represent 95% confidence intervals for both median triglyceride percent change from baseline (y-axis; vertical error bars) and mean EPA plasma concentration percent change from baseline (x-axis; horizontal error bars). This data is consistent with the observation in all studies to-date that a greater increase in EPA concentrations in both plasma and RBCs was observed for subjects administered about 4 g per day of EPA compared to those administered about 2 g per day. In addition, median percent reductions in triglycerides (compared to baseline) were higher for subjects administered about 4 grams of EPA per day than for those administered about 2 g of EPA per day in both the ANCHOR study (described herein and also in U.S. Pat. No. 8,410,486, incorporated herein by reference) and also in the MARINE study (see, e.g., U.S. Pat. No. 8,357,677, incorporated herein by reference).

What is claimed is:

1. A pharmaceutical composition comprising about 1 g of ethyl eicosapentaenoate, the pharmaceutical composition providing a mean plasma Cₘₐₓ of about 154.9+/−49.4 μg/mL, a mean plasma AUC₀₋₂₄ₕ of about 2907+/−1160 μg·h/mL, a median plasma Tₘₐₓ of about 5 hours, and a mean plasma T_{1/2} of about 75.1+/−46.5 hours of ethyl eicosapentaenoate when administered orally twice per day to a human subject.

2. The pharmaceutical composition of claim 1, wherein the pharmaceutical composition further provides a mean plasma Cₘᵢₙ of about 101.0+/−50.4 μg/mL of ethyl eicosapentaenoate.

3. The pharmaceutical composition of claim 1, wherein the pharmaceutical composition further provides a mean plasma CL/F of about 776.4+/−256.9 mL/h of ethyl eicosapentaenoate.

4. The pharmaceutical composition of claim 1, wherein the pharmaceutical composition further provides a mean plasma V_{z}/F of about 79.8+/−62.6 L of ethyl eicosapentaenoate.

5. The pharmaceutical composition of claim 1, wherein the pharmaceutical composition further provides a mean RBC Cₘₐₓ of about 42.3+/−14.0 μg/mL, a mean RBC AUC₀₋₂₄ₕ of about 801.5+/−268.5 μg·h/mL, a median RBC Tₘₐₓ of about 12 hours, and a mean RBC T_{1/2} of about 683.8 hours of ethyl eicosapentaenoate.

6. The pharmaceutical composition of claim 1, wherein the pharmaceutical composition further provides a mean RBC Cₘᵢₙ of about 31.2+/−12.2 μg/mL of ethyl eicosapentaenoate.

7. The pharmaceutical composition of claim 1, wherein the pharmaceutical composition further provides a mean plasma Cₘₐₓ of about 0.66+/−0.34 μg/mL, a mean plasma AUC₀₋₂₄ₕ of about 6.9+/−3.1 μg·h/mL, a median plasma Tₘₐₓ of about 5 hours, and a mean plasma T_{1/2} of about 80.8+/−37.5 hours of unesterified eicosapentaenoic acid when administered orally twice per day to a human subject.

8. The pharmaceutical composition of claim 1, wherein the pharmaceutical composition further provides a mean plasma Cₘᵢₙ of about 0.41+/−0.28 μg/mL of unesterified eicosapentaenoic acid.

9. The pharmaceutical composition of claim 1, wherein the pharmaceutical composition further provides a mean plasma CL/F of about 364,513+/−214,003 mL/h of unesterified eicosapentaenoic acid.

10. The pharmaceutical composition of claim 1, wherein the pharmaceutical composition further provides a mean plasma V_{z}/F of about 33,164+/−8,068 L of unesterified eicosapentaenoic acid.

11. A pharmaceutical composition comprising about 2 g of ethyl eicosapentaenoate, the pharmaceutical composition providing a mean plasma Cₘₐₓ of about 347.2+/−112.5 μg/mL, a mean plasma AUC₀₋₂₄ₕ of about 6519+/−1963 μg·h/mL, a median plasma Tₘₐₓ of 5 hours, and a mean plasma T_{1/2} of about 89.3+/−42.0 hours of ethyl eicosapentaenoate when administered orally twice per day to a human subject.

12. The pharmaceutical composition of claim 11, wherein the pharmaceutical composition further provides a mean plasma Cₘᵢₙ of about 101.0+/−50.4 μg/mL of ethyl eicosapentaenoate.

13. The pharmaceutical composition of claim 11, wherein the pharmaceutical composition further provides a mean plasma CL/F of about 683.7+/−280.6 mL/h of ethyl eicosapentaenoate.

14. The pharmaceutical composition of claim 11, wherein the pharmaceutical composition further provides a mean plasma V_{z}/F of about 88.4+/−55.2 L of ethyl eicosapentaenoate.

15. The pharmaceutical composition of claim 11, wherein the pharmaceutical composition further provides a mean RBC Cₘₐₓ of about 76.7+/−25.2 μg/mL, a mean RBC AUC₀₋₂₄ₕ of about 1472+/−469.5 μg·h/mL, a median RBC Tₘₐₓ of about 8 hours, and a mean RBC T_{1/2} of about 371.4+/−311.5 hours of ethyl eicosapentaenoate.

16. The pharmaceutical composition of any one of claim 11, wherein the pharmaceutical composition further provides a mean RBC Cₘᵢₙ of 64.1+/−21.8 μg/mL of ethyl eicosapentaenoate.

17. The pharmaceutical composition of claim 11, wherein the pharmaceutical composition further provides a mean plasma Cₘₐₓ of about 1.4+/−0.41 μg/mL, a mean plasma AUC₀₋₂₄ₕ of about 18.4+/−4.6 μg·h/mL, a median plasma Tₘₐₓ of about 5 hours, and a mean plasma T_{1/2} of about 97.2+/−36.5 hours of unesterified eicosapentaenoic acid.

18. The pharmaceutical composition of claim 11, wherein the pharmaceutical composition further provides a mean plasma Cₘᵢₙ of about 1.06+/−0.56 μg/mL of unesterified eicosapentaenoic acid.

19. The pharmaceutical composition of claim 11, wherein the pharmaceutical composition further provides a mean plasma CL/F of about 234,329+/−81,639 mL/h of unesterified eicosapentaenoic acid.

20. The pharmaceutical composition of claim 11, wherein the pharmaceutical composition further provides a mean plasma V_{z}/F of about 28,200+/−10,929 L of unesterified eicosapentaenoic acid.

21. The pharmaceutical composition of any claim 11, wherein the pharmaceutical composition is administered in unit dose form, wherein each unit dose includes about 1 g of ethyl eicosapentaenoate.
